(19) United States
(12) Patent Application Publication
(10) Pub. No.: US 2024/0277873 A1
(43) Pub. Date: Aug. 22, 2024

(54) ULTRABRIGHT CHEMILUMINESCENT PROBES FOR DETECTION AND IMAGING

(71) Applicant: Nanyang Technological University, Singapore (SG)

(72) Inventors: Kanyi PU, Singapore (SG); Jingsheng HUANG, Singapore (SG)

(21) Appl. No.: 18/566,471

(22) PCT Filed: Aug. 11, 2022
(86) PCT No.: PCT/SG2022/050572

§ 371 Date: Dec. 1, 2023

(30) Foreign Application Priority Data
Aug. 11, 2021 (SG) 10202108747Q

(51) Int. Cl. A61K 49/00

(52) U.S. Cl. CPC A61K 49/0021; A61K 49/0008; A61K 49/0054; A61K 49/0056

(57) ABSTRACT

Disclosed herein is a compound of Formula I, or pharmaceutically acceptable salt or solvate thereof. Also disclosed herein are methods for detection of neutrophil elastase in an analyte, detection of neutrophil elastase in vivo, identifying a compound suitable for the treatment of psoriasis, and identifying a compound suitable for the treatment of peritonitis.

### FIELD OF INVENTION

The current invention relates to chemiluminescent probes that are particularly suited for use in in vivo imaging techniques.

### BACKGROUND

The listing or discussion of a prior-published document in this specification should not necessarily be taken as an acknowledgment that the document is part of the state of the art or is common general knowledge.

Sensitive and dynamic detection of immune cells are imperative for understanding of their pathophysiological functions, exploration of their diagnostic and prognostic potentials, and innovation of treatment for cancer and autoimmune diseases. As a crucial player in innate immunity, neutrophils initiate the immediate actions by producing a variety of cytokines to manipulate the host-pathogen interactions in both acute and chronic inflammations including trauma, infection, and cancers. However, clinical methods for neutrophil detection depend on tissue biopsy or blood analysis, which are invasive and static.

Although molecular imaging offers a non-invasive and dynamic way for real-time tracking of neutrophils, most of them are antibody-conjugated imaging agents and thus inevitably encounter nonspecific binding with normal cells and strong background signals. Superior to these “always-on” probes, a few activatable fluorescence probes that only trigger their signals in the presence of neutrophil-overexpressed biomarkers have been recently synthesized, which showed increased selectivity and sensitivity. However, the requirement of real-time light excitation for fluorescence probes causes tissue autofluorescence and shallow tissue penetration, constraining their in vivo imaging applications.

Distinct from fluorescence imaging, chemiluminescence imaging eliminates the need of light excitation and thus avoids tissue background signal, and represents a more sensitive way of in vivo imaging of neutrophils. Among the many chemiluminescent substrates (luminol, acridine, etc.), Schaap's adamantylidene-1,2-dioxetane substrates can be modified into activatable chemiluminescence probes that only emit light in the presence of the biomarker of interest. Despite the ability to specifically detect various biomarkers including enzymes, small chemical molecules and reactive oxygen species (ROS), adamantylidene-1,2-dioxetane based probes suffer from short emission wavelengths, low aqueous chemiluminescence quantum yields (QY), and short half-lives. Recently, it was reported that the introduction of electron-withdrawing groups on the ortho position of phenol in adamantylidene-1,2-dioxetane increased the chemiluminescence QY to 0.023 einsteins/mol which is 3000-fold higher than the unmodified substrate. More recently, Huang et al. reported a chemiluminescent probe with a long near-infrared (NIR) emission at 780 nm by introducing Se atom into the acceptor skeleton of phenoxy-dioxetane substrates (Huang, J. et al., Angew. Chem. Int. Ed. 2021, 60, 3999-4003).

Nevertheless, there exists a need to discover new molecular design strategies to increase the half-lives of chemiluminescence probes to facilitate longitudinal tracking and real-time monitoring of neutrophils.

### SUMMARY OF INVENTION

Aspects and embodiments of the invention will now be discussed by reference to the following numbered embodiments.

1. A compound of formula I:

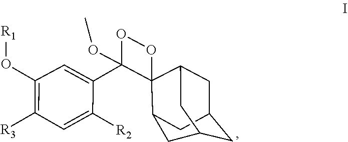

where:
R¹ represents CF₃S(O)₂ or

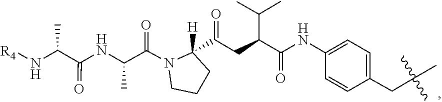

where the wiggly line represents the point of attachment to the rest of the molecule;
R₂ represents H, an acceptor group capable of red-shifting the chemiluminescence emission to the near-infrared region, a polyethylene glycol group, a halogen atom, an electron-withdrawing group or a π* acceptor group capable of accepting electrons; R₃ represents H, an acceptor group capable of red-shifting the chemiluminescence emission to the near-infrared region, a polyethylene glycol group, a halogen atom, an electron-withdrawing group, a π* acceptor group capable of accepting electrons, or

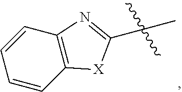

where X represents Se, or more particularly S or O and the wiggly line represents the point of attachment to the rest of the molecule;
R₄ represents

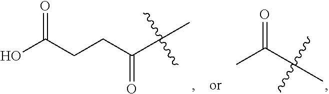

where the wiggly lines represent the point of attachment to the rest of the molecule or a pharmaceutically acceptable salt or solvate thereof.

2. The compound, or pharmaceutically acceptable salt or solvate thereof, according to Clause 1, wherein each acceptor group capable of red-shifting the chemiluminescence emission to the near-infrared region is independently selected from the list of:

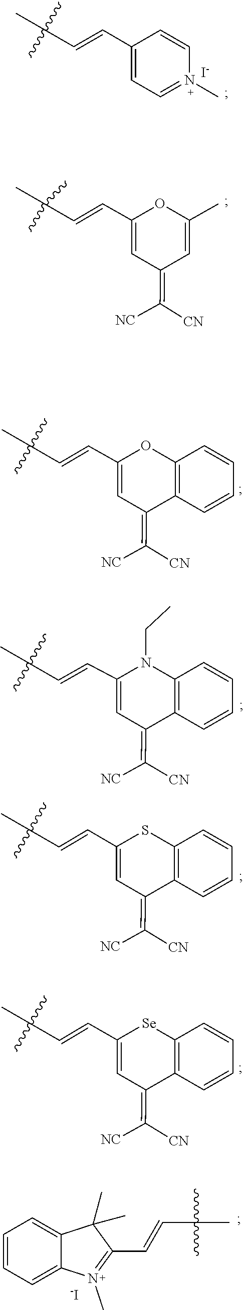

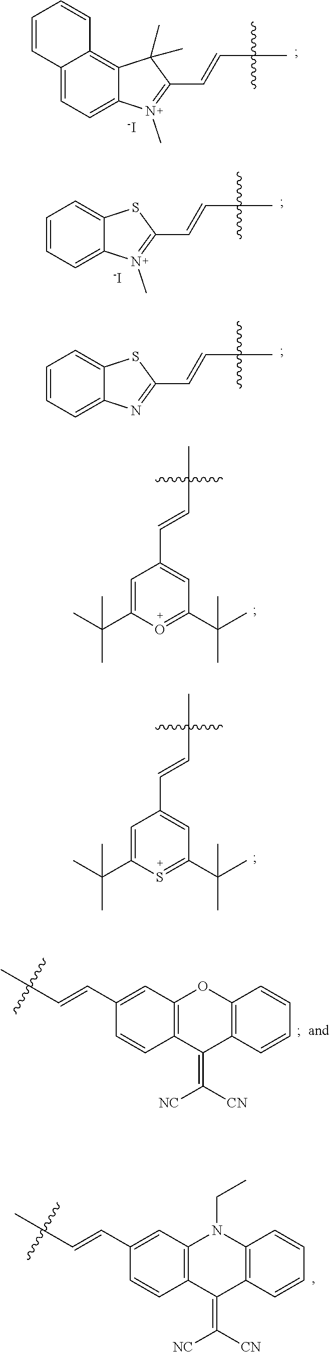

where the wavy line represents the point of attachment to the rest of the molecule, optionally wherein each acceptor group capable of red-shifting the chemiluminescence emission to the near-infrared region is

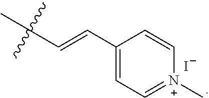

3. The compound, or pharmaceutically acceptable salt or solvate thereof, according to Clause 1 or Clause 2, wherein each π* acceptor group capable of accepting electrons is selected from the list:

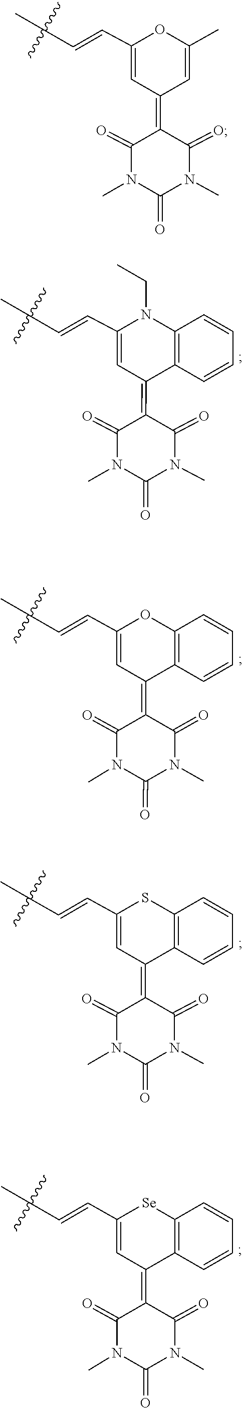 [SMILES: */C=C/C1=CC(=C2C(=O)N(C)C(=O)N(C)C2=O)C=C(C)O1 ; */C=C/C1=CC(=C2C(=O)N(C)C(=O)N(C)C2=O)c2ccccc2N1CC ; */C=C/C1=CC(=C2C(=O)N(C)C(=O)N(C)C2=O)c2ccccc2O1 ; */C=C/C1=CC(=C2C(=O)N(C)C(=O)N(C)C2=O)c2ccccc2S1 ; */C=C/C1=CC(=C2C(=O)N(C)C(=O)N(C)C2=O)c2ccccc2[Se]1]

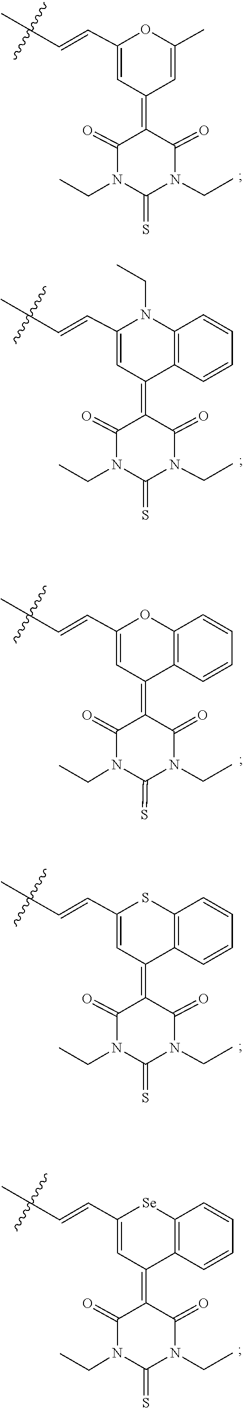

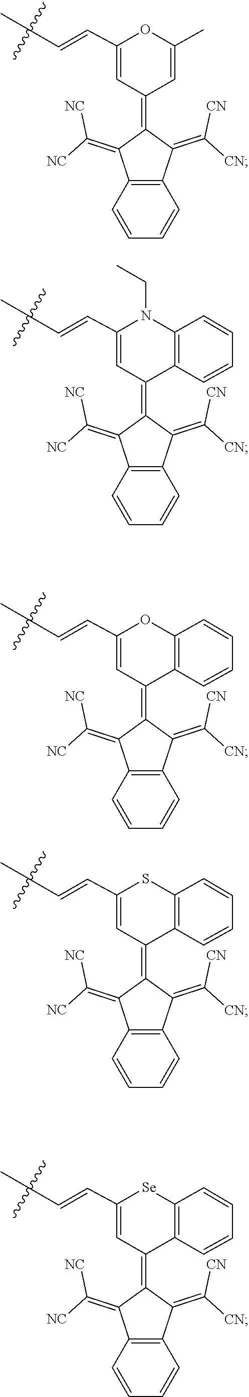

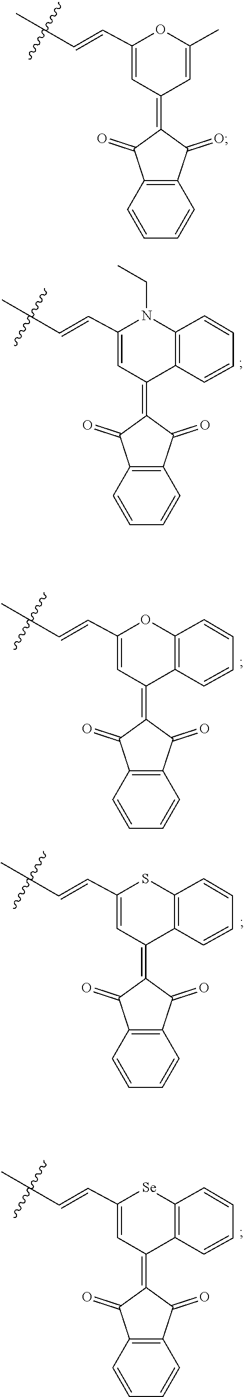

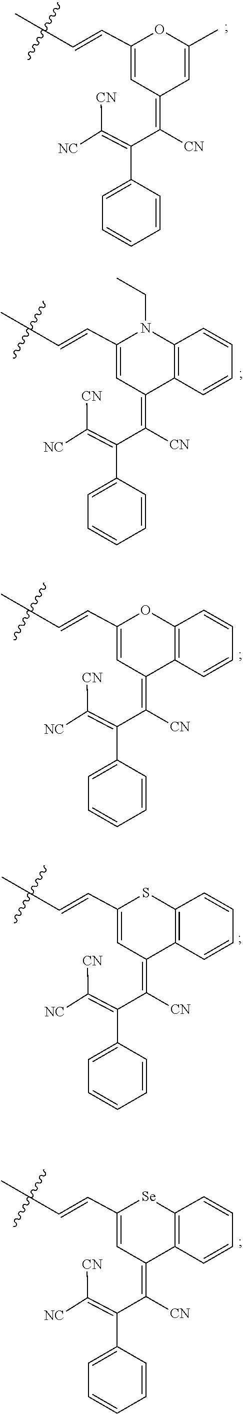

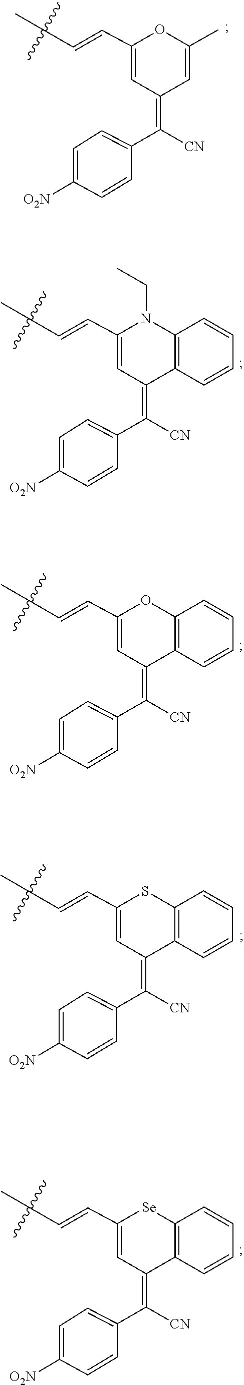 [SMILES: */C=C/C1=CC(=C(/C#N)c2ccc([N+](=O)[O-])cc2)/C=C(C)O1 ; */C=C/C1=CC(=C(/C#N)c2ccc([N+](=O)[O-])cc2)/c2ccccc2N1CC ; */C=C/C1=CC(=C(/C#N)c2ccc([N+](=O)[O-])cc2)/c2ccccc2O1 ; */C=C/C1=CC(=C(/C#N)c2ccc([N+](=O)[O-])cc2)/c2ccccc2S1 ; */C=C/C1=CC(=C(/C#N)c2ccc([N+](=O)[O-])cc2)/c2ccccc2[Se]1]

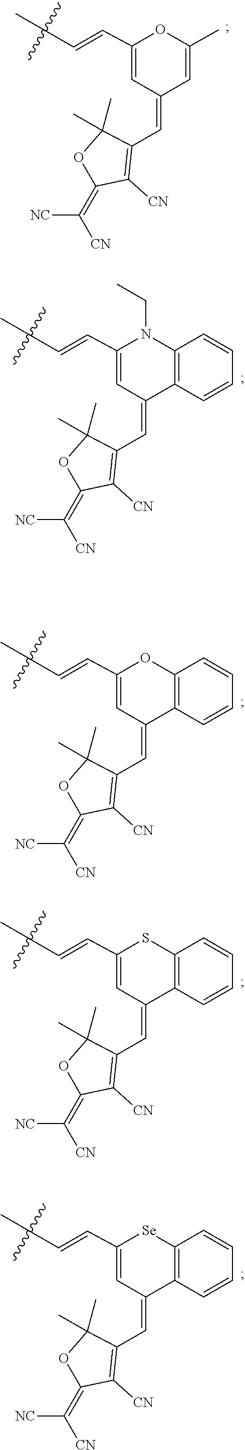

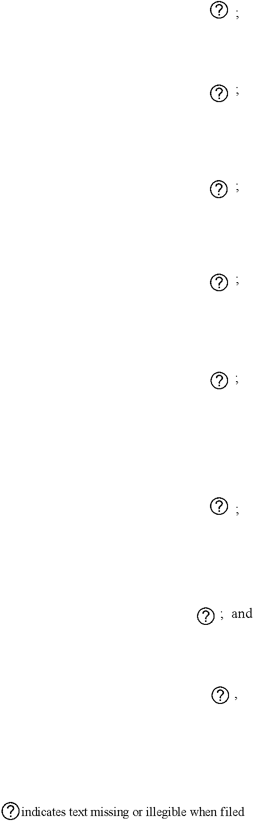

where the wavy line represents the point of attachment to the rest of the molecule.

4. The compound, or pharmaceutically acceptable salt or solvate thereof, according to any one of the preceding clauses, wherein each electron withdrawing group is selected from the list:

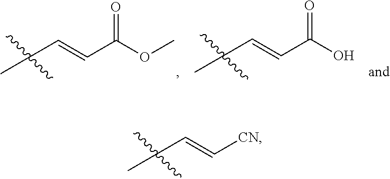

where the wavy line represents the point of attachment to the rest of the molecule.

5. The compound, or pharmaceutically acceptable salt or solvate thereof, according to any one of the preceding clauses, wherein each polyethylene glycol group has the formula:

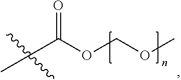

where n is from 1 to 227 and the wiggly line represents the point of attachment to the rest of the molecule.

6. The compound, or pharmaceutically acceptable salt or solvate thereof, according to any one of the preceding claims, wherein:
R₁ is

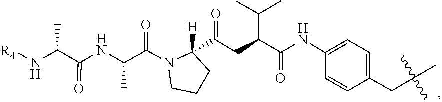

where the wiggly line represents the point of attachment to the rest of the molecule;
R₂ represents H, an acceptor group capable of red-shifting the chemiluminescence emission to the near-infrared region, or a polyethylene glycol group;
R₃ represents

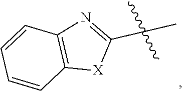

where X represents S or O and the wiggly line represents the point of attachment to the rest of the molecule.

7. The compound according to Clause 6, wherein R₁ represents CF₃S(O)₂.

8. The compound according to Clause 6 or Clause 7, wherein R₂ represents H or

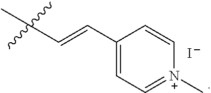

9. The compound, or pharmaceutically acceptable salt or solvate thereof, according to any one of Clauses 1 to 5, wherein:
R¹ represents

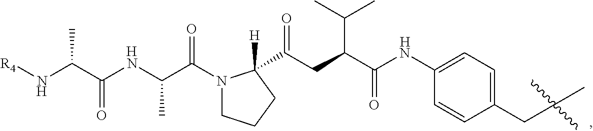

where the wiggly line represents the point of attachment to the rest of the molecule;
R₂ represents H, a halogen atom, an electron-withdrawing group or a π* acceptor group capable of accepting electrons;
R₃ represents H, a halogen atom, an electron-withdrawing group or a π* acceptor group capable of accepting electrons.

10. The compound according to Clause 9, wherein R₃ represents H, a halogen atom, or an electron-withdrawing group.

11. The compound, or pharmaceutically acceptable salt or solvate thereof, according to Clause 1 wherein the compound is selected from the list:

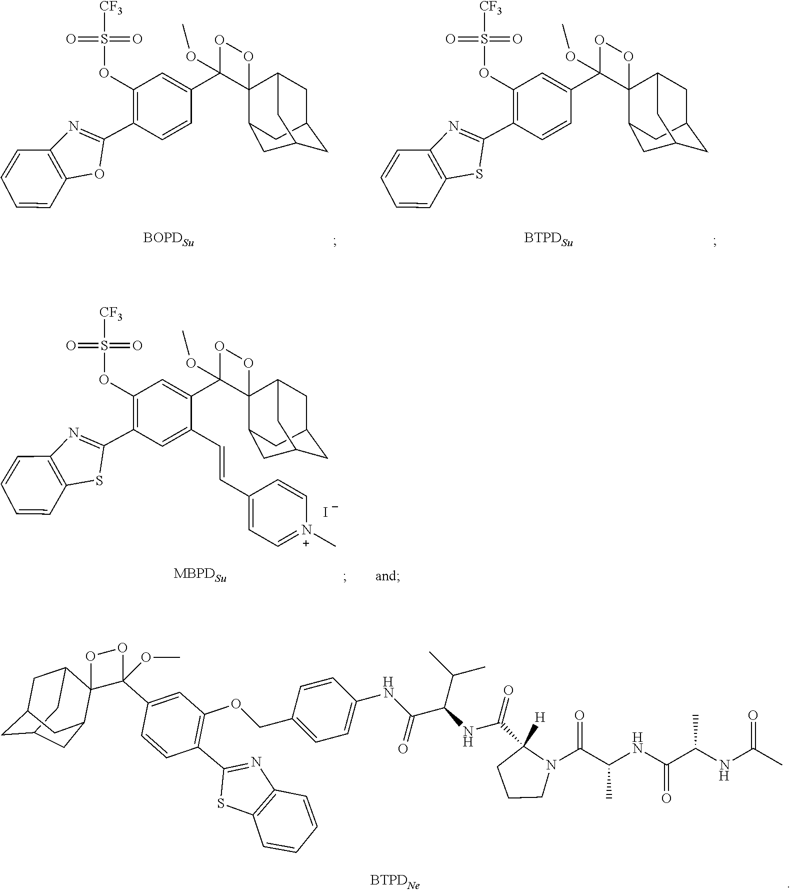

12. A method for detection of neutrophil elastase in an analyte, the method comprising the following steps:
(a) providing an analyte and a fluid comprising a compound of formula I, or pharmaceutically acceptable salt or solvate thereof, according to any one of Clauses 1 to 11;
(b) contacting the analyte with the fluid comprising a compound of formula I, or pharmaceutically acceptable salt or solvate thereof, for a period of time; and
(c) after the period of time detecting any chemiluminescence, wherein the presence of neutrophil elastase in the fluid comprising the analyte and the fluid comprising a compound of formula I, or pharmaceutically acceptable salt or solvate thereof, is indicated by chemiluminescence.

13. A method for detection of neutrophil elastase in vivo, the method comprising the following steps:
(ai) administering a compound of formula I, or pharmaceutically acceptable salt or solvate thereof, according to any one of Clauses 1 to 11 to a subject; and
(aii) detecting any chemiluminescence, wherein the presence of neutrophil elastase in vivo is indicated by chemiluminescence.

14. A method for identifying a compound suitable for the treatment of psoriasis, the method comprising:
(bi) providing a mouse where the skin exhibits neutrophil infiltration (e.g. where the dorsal skin of the mouse has been treated by imiquimod);
(bii) contacting the skin with a test material for a first period of time;
(biii) after the first period of time, contacting the drug-treated skin with a compound of formula I, or a pharmaceutically acceptable salt or solvate thereof, as defined in any one of Clauses 1 to 11 for a second period of time; and
(biv) after the second period of time any chemiluminescent signal skin is detected and compared to a blank control, where a reduced quantitative chemiluminescence readout in the presence of the test compound compared to the blank control is indicative of anti-psoriasis activity.

15. A method for identifying a compound suitable for the treatment of peritonitis, the method comprising:
(ci) providing a mouse suffering from peritonitis, where the peritonitis is associated with ascites, where the ascites exhibits neutrophil infiltration (e.g where the mouse's peritonitis was induced by intraperitoneal injection of lipopolysaccharides (LPS));
(cii) contacting an abdomen of the mouse with a test material for a first period of time;
(ciii) after the first period of time, contacting the LPS-treated abdomen with a compound of formula I, or a pharmaceutically acceptable salt or solvate thereof, as defined in any one of Clauses 1 to 11 for a second period of time; and
(civ) after the second period of time any chemiluminescent signal abdomen is detected and compared to a blank control, where a reduced quantitative chemiluminescence readout in the presence of the test compound compared to the blank control is indicative of peritonitis activity,
where mice were treated with PBS only.

### DRAWINGS

FIG. 1 depicts (a) synthetic route for O₂^{•-}-responsive chemiluminescent probes (BOPD_{Su}, BTPD_{Su}, and MBPD_{Su}): (i) Pd(OAc)₂, PPh₃, CuI, Cs₂CO₃, dimethylacetamide, 145° C., 6 h, 38% yield (compounds 3-0), 31% yield (compounds 3-S), 41% yield (compound 4); (ii) compound 3-X (X=O or S), Tf₂O, pyridine, dichloromethane (DCM), 0° C., 2 h, N₂, 95% (5-0), 91% (5-S); (iii) piperidine, compound 4, acetonitrile (ACN), N₂, reflux, 3 h, 68% yield (compound 6); (iv) compound 5-X (X=O or S), methylene blue, DCM, 0° C., 6 h, white light, 91% yield (BOPD_{Su}) and 95% (BTPD_{Su}); (v) compound 6, Tf₂O, DCM, 0° C., 2 h, N₂, without further purification; and (vi) methylene blue, DCM, 0° C., 6 h, white light, air, 92% (MBPD_{Su}); (b) synthetic route for chemiluminescent probe BTPD_{Ne} for neutrophil elastase (NE) detection: (vii) peptide (AAPV), p-aminobenzyl alcohol, 2-ethoxy-1-ethoxycarbonyl-1,2-dihydroquinoline (EEDQ), DCM, room temperature (r.t.), 6 h, 87% yield; (viii) compound 7, PBr₃, tetrahydrofuran (THF), 0° C., 3 h, without further purification; (ix) crude product 8, 3-S, N,N-diisopropylethylamine (DIPEA), ACN, 60° C., overnight, 23% yield; (x) compound 9, methylene blue, DCM, 0° C., 6 h, white light, air, 96% yield; (xi) crude product 8, green chemiluminophere precursor, DIPEA, ACN, 60° C., overnight, 44% yield; and (xii) compound 10, methylene blue, DCM, 0° C., 6 h, white light, air, 92% yield; (c) synthetic route for activated probes ABOPD, ABTPD, and AMBPD: (xiii) benzothiazole or benzoxazole, Pd(OAc)₂, PPh₃, CuI, Cs₂CO₃, DMAc, 145° C., 6 h, 58% yield (ABOPD), 45% yield (ABTPD); and (xiv) compound 6, K₂CO₃, methylene blue, 0° C., 6 h, white light, air, methanol (MeOH), 67% yield; and (d) synthetic route for activated chemiluminescence intermediate BTPD: (xv) 3-S, methylene blue, DCM, 0° C., 6 h, white light, air, 94% yield.

FIG. 2 depicts (a) ultraviolet (UV) absorption of unactivated probes (BOPD_{Su}, BTPD_{Su} and MBPD_{Su}, 20 μM) in absence and presence of KO₂ (50 μM) in phosphate buffered saline (PBS, 10 mM, pH 7.4, 10% dimethylsulfoxide (DMSO)), respectively; and (b) UV and (c) fluorescence spectra of activated probes (ABOPD, ABTPD, AMBPD and AMPD, 20 μM) in PBS (10 mM, pH 7.4, 10% DMSO), respectively.

FIG. 3 depicts (a) fluorescence changes of BOPD_{Su}, BTPD_{Su}, MBPD_{Su} and MPD_{Su} (20 μM) in presence of different RONS (40 μM) or other metal ions (100 μM) in PBS (10 mM, pH 7.4, 10% DMSO) at 37° C. with 10 s of response time; and (b) limit of detection (LOD) of BOPD_{Su}, BTPD_{Su}, and MBPD_{Su} and MPD_{Su} toward KO₂ in PBS (10 mM, pH 7.4, 10% DMSO) at 37° C. Data are the mean±SD, n=3 independent experiments.

FIG. 4 depicts (a) chemical structures of BOPD_{Su}, BTPD_{Su}, MBPD_{Su} and MPD_{Su}; (b) chemiluminescence spectra and (d) half-lives of BOPD_{Su}, BTPD_{Su} and MBPD_{Su} and MPD_{Su} (20 μM) in the presence of O₂^{•-} (40 μM) in PBS (10 mM, pH 7.4, 10% DMSO) at 37° C.; (c) chemiluminescence changes of BOPD_{Su}, BTPD_{Su} and MBPD_{Su} and MPD_{Su} after incubation with different reactive oxygen and nitrogen species (RONS) (40 μM) or other metal ions (100 μM) in PBS (10 mM, pH 7.4) at 37° C. for 10 s. Data are the mean±SD, n=3 independent experiments; (e)¹H nuclear magnetic resonance (NMR) spectra of ABTPD and AMPD in CD₃CN with the incremental addition of DMSO-d₆; and (f) chemical shift changes of proton of phenolic hydroxyl of ABTPD and AMPD in CD₃CN with DMSO-d₆ increase.

FIG. 5 depicts high performance liquid chromatography (HPLC) analysis of (a) BOPD_{Su}, (b) BTPD_{Su} and (c) MBPD_{Su} (20 μM) before and after cleavage by O₂^{•-} (50 μM) in PBS (10 mM, pH=7.4, 10% DMSO) at 37° C.

FIG. 6 depicts chemiluminescent probe for neutrophils detection. (a) Mechanism of BTPD_{Ne} for NE activation; (b) Chemiluminescence spectrum of BTPD_{Ne} (20 μM) in the absence and presence of NE (0.1 U/ml) in 50 mM Tris, 1 M NaCl, 0.05% (w/v) Brij-35, pH 7.5; (c) HPLC analysis of BTPD_{Ne} after 30 min incubation; (d) Time course of BTPD_{Ne} and MPD_{Ne} chemiluminescence intensities in the presence of NE; (e) Chemiluminescence imaging of neutrophil, dendritic cells (DC), macrophage (Mac), cytotoxic T lymphocyte (CTL) and 3T3 cells after incubation with BTPD_{Ne} (20 μM) for 60 min; (f) Chemiluminescence imaging of neutrophil after incubation with BTPD_{Ne} and MPD_{Ne} (10 μM) for 30 and 60 min; (g) Quantification of signal enhancement in (e); and (h) Quantification of signal intensity in (f).

FIG. 7 depicts probe MPD_{Ne} for in vitro detection of NE activity. (a) Responsive mechanism illustrations of MPD_{Ne} for NE activation; (b) Chemiluminescence spectrum of MPD_{Ne} in the presence of NE (0.1 U/ml) in 50 mM Tris, 1 M NaCl, 0.05% (w/v) Brij-35, pH 7.5 after 30 min incubation; (c) HPLC analysis of MPD_{Ne} after 30 min incubation; and (d) Chemiluminescence changes of MPD_{Ne} (20 μM) in the presence of NE (0.1 U/ml), other different enzymes (Alanine aminopeptidase (APN), Alkaline Phosphatase (ALP), Caspase 3 (Cas-3), Cathepsin B (Cat B), γ-Glutamyl Transferase (GGT), furine, β-galactosidase (β-gal), nitroreductase (NTR), cathepsin G (CatG), and proteinase 3 (PR3)) (˜0.1 U/mL).

FIG. 8 depicts chemiluminescence and fluorescence changes of BTPD_{Ne} (20 μM) in the presence of NE (0.1 U/ml), other different enzymes (˜0.1 U/mL). Inset images: the corresponding chemiluminescence images. Other different enzymes: APN, ALP, Cas-3, cat B, GGT, furin, β-gal, NTR, CatG, and PR3.

FIG. 9 depicts LC-MS analysis of HPLC eluent peak at 15.9 min in FIG. 6c.

FIG. 10 depicts enzyme kinetics study of 0.1 U/mL NE with BTPD_{Ne} ranging from 2 to 80 μM. K_{M}=52.17 μM, K_{cat}=1.15 s⁻¹, K_{cat}/K_{M}=0.022 μM⁻¹·s⁻¹.

FIG. 11 depicts chemiluminescence pictures of BTPD_{Su} (100 μM) after treatment with O₂^{•-} (4 eq.) in PBS (10 mM, pH=7.4, 10% DMSO) at 37° C.

FIG. 12 depicts the mechanistic comparison of the chemiluminescence benzoazole-phenoxyl-dioxetane substrates (b) with the reported Schaap's dioxetane with electron-withdrawing groups (a). CIEEL: chemically initiated electron exchange luminescence.

FIG. 13 depicts the half-lives of BTPD_{Su} (20 μM) and BTPD (20 μM) in pure DMSO upon addition of excess O₂^{•-} (100 μM) and in 10 mM PBS (10% DMSO, pH 7.4) at 37° C., respectively.

FIG. 14 depicts the (a) stability of BTPD_{Su} (20 μM) in different pH buffers with 10% DMSO for 2 h incubation at 37° C.; (b) fluorescence intensity of ABTPD in different pH buffers with 10% DMSO; and (c) time-course and (d) half-lives of BTPD_{Su} (20 μM) (a) upon addition of excess O₂^{•-} (40 μM) in different pH buffers with 10% DMSO at 37° C.

FIG. 15 depicts chemiluminescence intensities of BTPD_{Ne} and MPD_{Ne} (20 μM) after incubation for 30 min in 10 mM PBS (pH 7.4) and healthy mouse blood (100 μL), respectively.

FIG. 16 depicts cell viability of 3T3 and neutrophil after incubation with BTPD_{Ne} at different concentrations ranging from 2.5 to 50 μM for 24 h. All data are the mean±SD, n=3.

FIG. 17 depicts (a) schematic illustration for the mechanism of neutrophil infiltration in lipopolysaccharides (LPS)-induced peritoneal and sensing mechanism of BTPD_{Ne}; (b) chemiluminescent images of LPS-treated mice were acquired at 0, 5, 10, 20, 45, and 60 min after intraperitoneal injection of BTPD_{Ne} and MPD_{Ne} (40 μM·kg⁻¹). The control group: PBS; (c) quantification of chemiluminescent signals in FIG. 17b; (d) flow cytometry analysis of peritoneal fluid from mice in control and LPS-treated groups; and (e) quantification of chemiluminescent signals in FIG. 17d. (***p<0.001).

FIG. 18 depicts (a) chemiluminescent images of PBS-treated mice acquired at 0, 5, 10, 20, 45, and 60 min after intraperitoneal injection of BTPD_{Ne} and MPD_{Ne} (40 μM·kg⁻¹). The control group: PBS; and (b) quantification of chemiluminescent signals in FIG. 18a.

FIG. 19 depicts in vivo real-time chemiluminescence imaging of neutrophils in the mouse model of IMQ-induced psoriasis. (a) Schematic illustration for the mechanism of neutrophil infiltration in IMQ-induced psoriasis, microneedle-assisted delivery and sensing mechanism of BTPD_{Ne}; (b) Chemiluminescent images of mice were acquired at 0, 3, and 30 min after dermal topical administration of BTPD_{Ne} (10 μL, 1 mM in DMSO) for 1, 2, and 3 days. The control group: PBS. The inhibition group: CsA (20 mg kg⁻¹) once a day for 2 days after IMQ-treatment; (c) Quantification of chemiluminescent signals in FIG. 19b. (**p<0.01); and (d) Histopathologic and immunohistochemical (arrows indicate neutrophils infiltration) and (e) Flow cytometry analyses of dorsal skins of mice in different groups. The white borders delineate the epidermis.

FIG. 20 depicts Pearson's correlation coefficient between chemiluminescence signal of in vivo imaging (FIG. 19b) and activated neutrophil number determined by flow cytometry (FIG. 19e) in peritonitis mouse models.

FIG. 21 depicts the immunohistochemical analysis of slides from dorsal skins of mice bearing psoriasis at day 2 post-treatment with IMQ. APC-Ly6G labeling mechanism: monoclonal antibody 1A8-Ly6G reacts with mouse Ly-6G which is a 25-kDa GPI protein expressed exclusively by neutrophils.

FIG. 22 depicts (a) fluorescence imaging of neutrophil, DC, Mac, CTL and 3T3 cells after incubation with BTPD_{Ne} (20 μM) for 30 min; and (b) quantification of signal enhancement in (a).

### DESCRIPTION

It has been surprisingly found that bright, activatable chemiluminescent probes with intramolecular hydrogen bonds can prolong the half-lives for in vivo imaging of neutrophils. Thus, in a first aspect of the invention there is provided a compound of formula I:

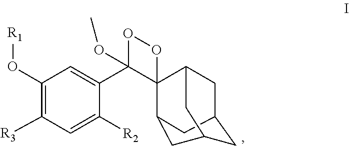

where:
R¹ represents CF₃S(O)₂ or

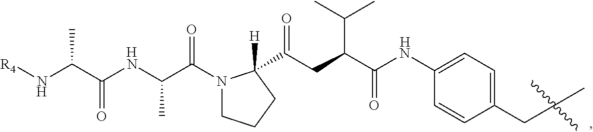

where the wiggly line represents the point of attachment to the rest of the molecule;
R₂ represents H, an acceptor group capable of red-shifting the chemiluminescence emission to the near-infrared region, a polyethylene glycol group, a halogen atom, an electron-withdrawing group or a π* acceptor group capable of accepting electrons;
R₃ represents H, an acceptor group capable of red-shifting the chemiluminescence emission to the near-infrared region, a polyethylene glycol group, a halogen atom, an electron-withdrawing group, a π* acceptor group capable of accepting electrons, or

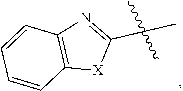

where X represents Se, or more particularly S or O and the wiggly line represents the point of attachment to the rest of the molecule;
R₄ represents

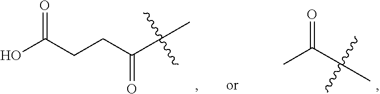

where the wiggly lines represent the point of attachment to the rest of the molecule or a pharmaceutically acceptable salt or solvate thereof.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

References herein (in any aspect or embodiment of the invention) to compounds of formula I include references to such compounds per se, to such compounds with intramolecular H-bonding, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well-known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Compounds of formula I, as well as pharmaceutically acceptable salts and solvates of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers with intramolecular H-bonding. All the forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively, the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

The term “halo”, when used herein, includes references to fluoro, chloro, bromo and iodo.

As noted above, the term “acceptor group” refers to a moiety that can red-shift the chemiluminescence emission of the compound of formula I to the near-infrared region. Examples of such acceptor groups include, but are not limited to moieties selected from the list of:

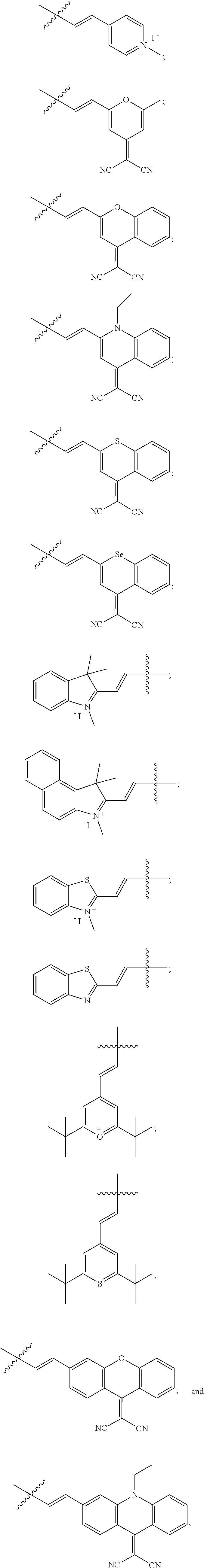

where the wavy line represents the point of attachment to the rest of the molecule. In particular embodiments of the invention that may be mentioned herein, each acceptor group capable of red-shifting the chemiluminescence emission to the near-infrared region may be

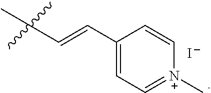

As noted above, the term “π* acceptor group” refers to a moiety that can accept electrons. Examples of such π* acceptor groups include, but are not limited to moieties selected from the list of:

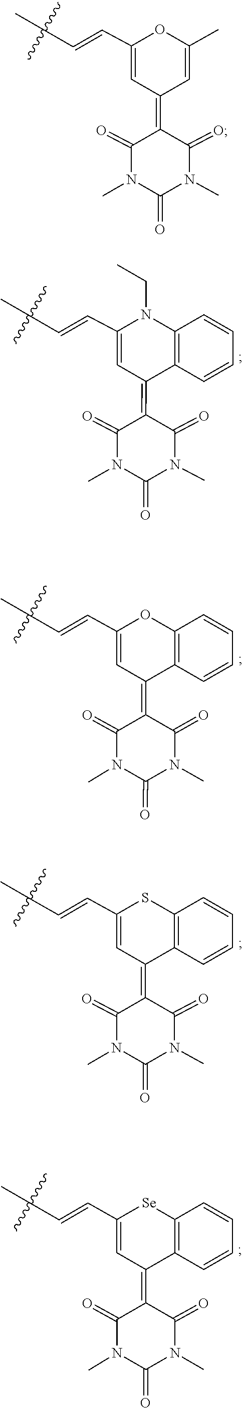 [SMILES: */C=C/C1=CC(=C2C(=O)N(C)C(=O)N(C)C2=O)C=C(C)O1 ; */C=C/C1=CC(=C2C(=O)N(C)C(=O)N(C)C2=O)c2ccccc2N1CC ; */C=C/C1=CC(=C2C(=O)N(C)C(=O)N(C)C2=O)c2ccccc2O1 ; */C=C/C1=CC(=C2C(=O)N(C)C(=O)N(C)C2=O)c2ccccc2S1 ; */C=C/C1=CC(=C2C(=O)N(C)C(=O)N(C)C2=O)c2ccccc2[Se]1]

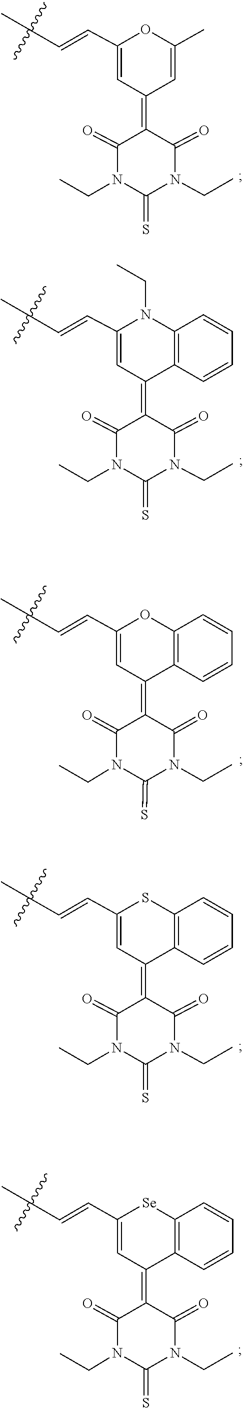

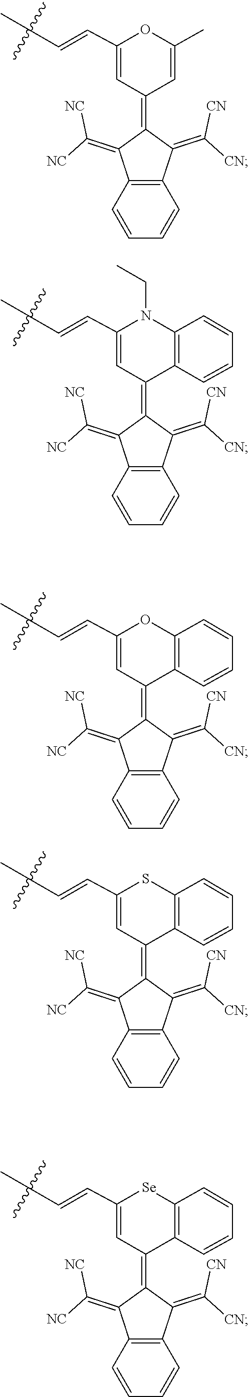

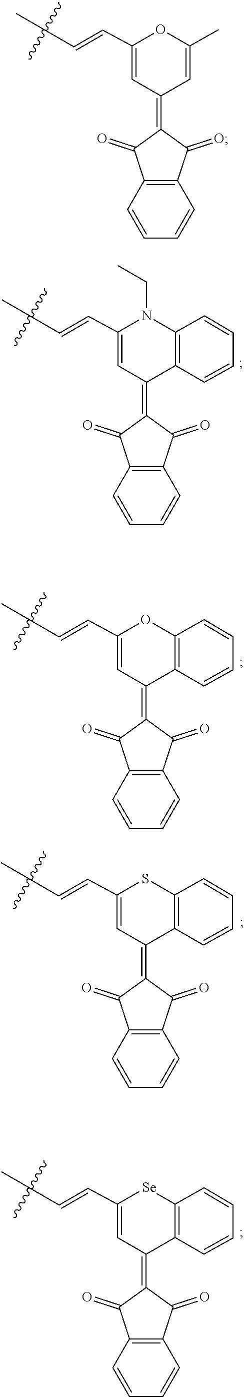

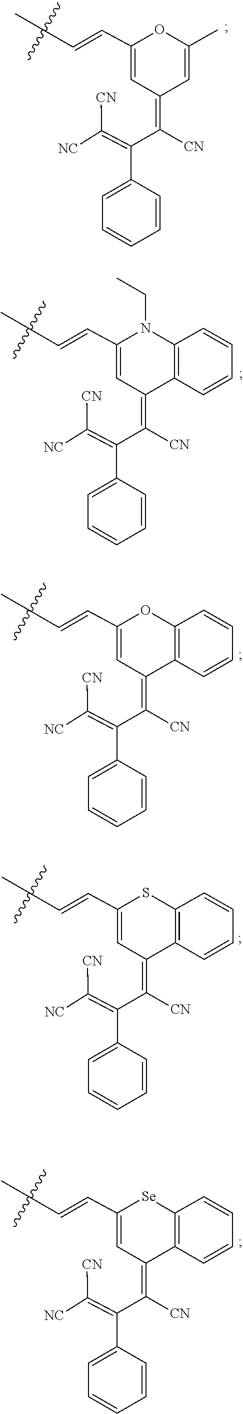

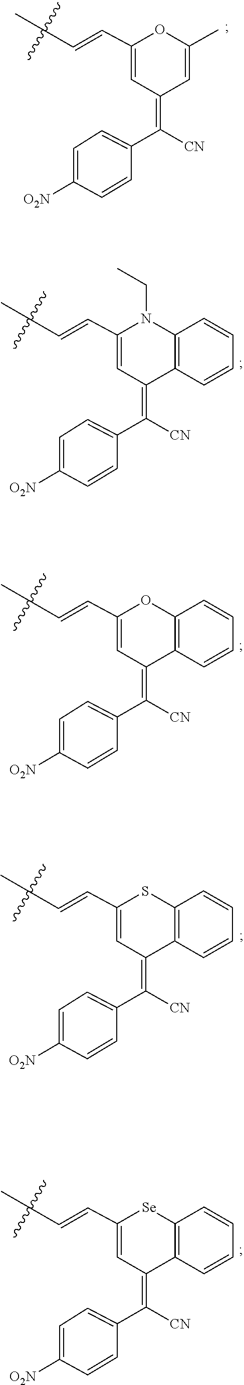 [SMILES: */C=C/C1=CC(=C(/C#N)c2ccc([N+](=O)[O-])cc2)/C=C(C)O1 ; */C=C/C1=CC(=C(/C#N)c2ccc([N+](=O)[O-])cc2)/c2ccccc2N1CC ; */C=C/C1=CC(=C(/C#N)c2ccc([N+](=O)[O-])cc2)/c2ccccc2O1 ; */C=C/C1=CC(=C(/C#N)c2ccc([N+](=O)[O-])cc2)/c2ccccc2S1 ; */C=C/C1=CC(=C(/C#N)c2ccc([N+](=O)[O-])cc2)/c2ccccc2[Se]1]

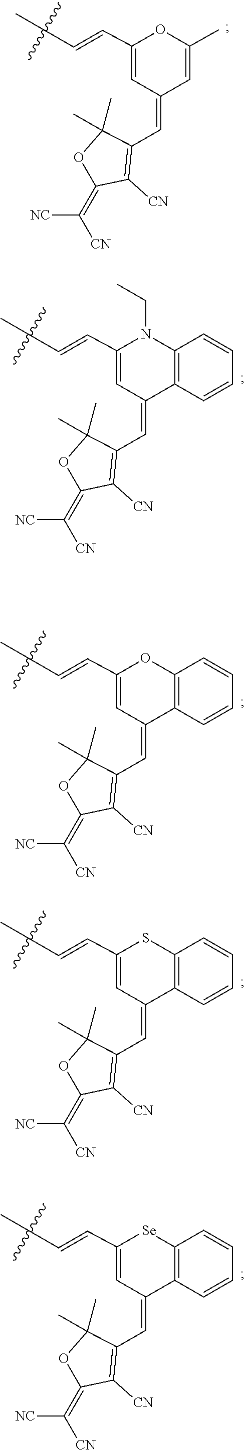

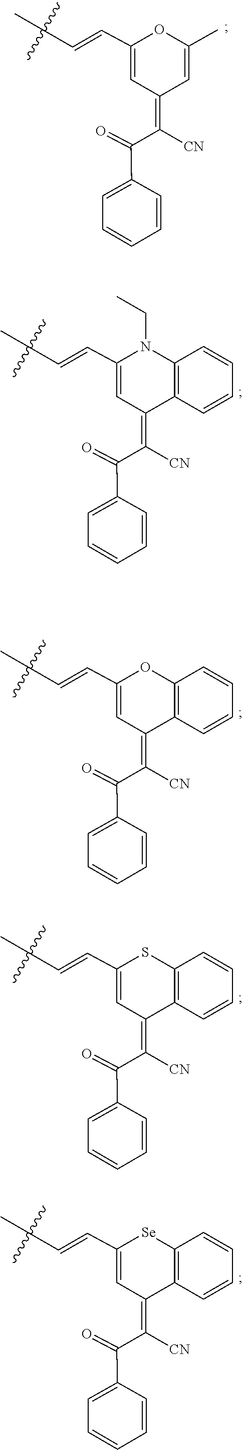 [SMILES: */C=C/C1=CC(=C(/C#N)C(=O)c2ccccc2)/C=C(C)O1 ; */C=C/C1=CC(=C(/C#N)C(=O)c2ccccc2)/c2ccccc2N1CC ; */C=C/C1=CC(=C(/C#N)C(=O)c2ccccc2)/c2ccccc2O1 ; */C=C/C1=CC(=C(/C#N)C(=O)c2ccccc2)/c2ccccc2S1 ; */C=C/C1=CC(=C(/C#N)C(=O)c2ccccc2)/c2ccccc2[Se]1]

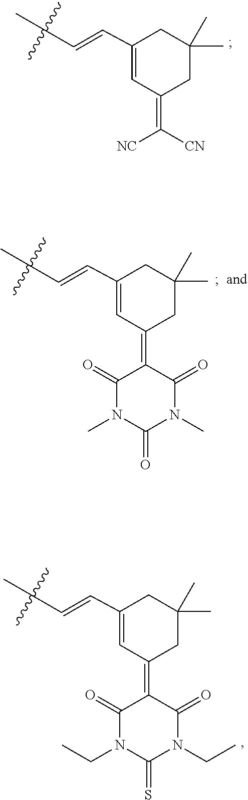 [SMILES: */C=C/C1=CC(=C(C#N)C#N)CC(C)(C)C1 ; */C=C/C1=CC(=C2C(=O)N(C)C(=O)N(C)C2=O)CC(C)(C)C1 ; */C=C/C1=CC(=C2C(=O)N(CC)C(=S)N(CC)C2=O)CC(C)(C)C1]

where the wavy line represents the point of attachment to the rest of the molecule.

Any suitable electron withdrawing group may be used as part of the compounds of formula I. Examples of suitable electron withdrawing groups include, but are not limited to a group selected from the list:

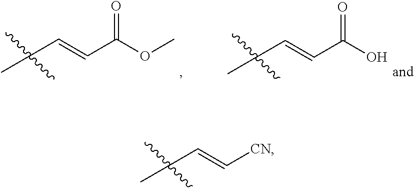

where the wavy line represents the point of attachment to the rest of the molecule.

In embodiments of the invention, each polyethylene glycol group may independently have the formula:

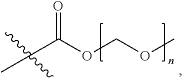

where n is from 1 to 227 and the wiggly line represents the point of attachment to the rest of the molecule.

As will be appreciated, R² and R³ may be the same or different. As such, in embodiments of the invention:
R₂ and R₃ may independently represent H, an acceptor group capable of red-shifting the chemiluminescence emission to the near-infrared region, a polyethylene glycol group, a halogen atom, an electron-withdrawing group or a π* acceptor group capable of accepting electrons;
R₂ represents H, an acceptor group capable of red-shifting the chemiluminescence emission to the near-infrared region, or a polyethylene glycol group, and R₃ represents

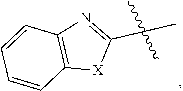

where X represents S or O and the wiggly line represents the point of attachment to the rest of the molecule;
R₂ and R₃ represent H, a halogen atom, an electron-withdrawing group or a π* acceptor group capable of accepting electrons; or
R₂ represents H, a halogen atom, an electron-withdrawing group or a π* acceptor group capable of accepting electrons and R₃ represents H, a halogen atom, or an electron-withdrawing group.

In particular embodiments that may be mentioned herein the compound of formula I, or a pharmaceutically acceptable salt or solvate thereof, may be one in which:
R₁ is

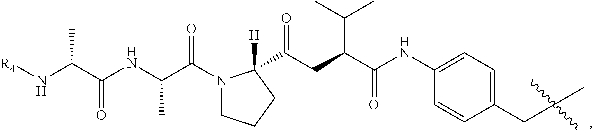

where the wiggly line represents the point of attachment to the rest of the molecule;
R₂ represents H, an acceptor group capable of red-shifting the chemiluminescence emission to the near-infrared region, and a polyethylene glycol group;
R₃ represents

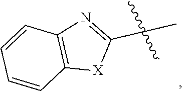

where X represents S or O and the wiggly line represents the point of attachment to the rest of the molecule. In more particular embodiments that may be mentioned herein of such embodiments, R₁ may represent CF₃S(O)₂ and/or R₂ may represent H or

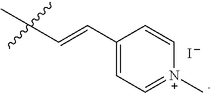

In alternative embodiments that may be mentioned herein, the compound of formula I or a pharmaceutically acceptable salt or solvate thereof, may be one in which R¹ represents

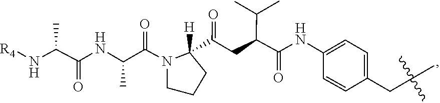

where the wiggly line represents the point of attachment to the rest of the molecule;
R₂ represents H, a halogen atom, an electron-withdrawing group or a π* acceptor group capable of accepting electrons; and
R₃ represents H, a halogen atom, an electron-withdrawing group or a π* acceptor group capable of accepting electrons. In more particular embodiments, R₃ may represent H, a halogen atom, or an electron-withdrawing group.

In particular embodiments of the invention, the compound of formula I may be selected from the list:

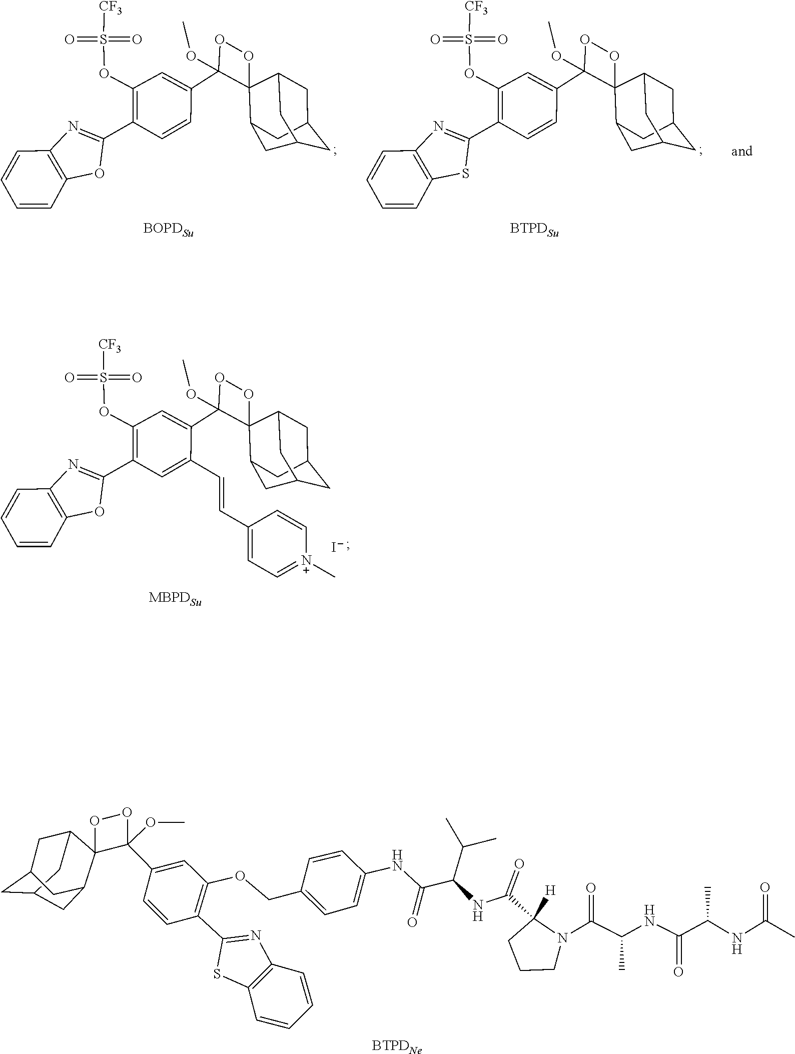

or pharmaceutically acceptable salts or solvates thereof.

In a further aspect of the invention, there is provided a method for detection of neutrophil elastase in an analyte, the method comprising the following steps:
(a) providing an analyte and a fluid comprising a compound of formula I, or pharmaceutically acceptable salt or solvate thereof, as described hereinbefore;
(b) contacting the analyte with the fluid comprising a compound of formula I, or pharmaceutically acceptable salt or solvate thereof, for a period of time; and
(c) after the period of time detecting any chemiluminescence, wherein the presence of neutrophil elastase in the fluid comprising the analyte and the fluid comprising a compound of formula I, or pharmaceutically acceptable salt or solvate thereof, is indicated by chemiluminescence.

The sample may be prepared and used as described in the examples section below. As will be appreciated, the skilled person may adapt the protocols disclosed below in line with their knowledge and the condition under consideration.

As neutrophil elastase is associated with inflammation and cancer, the detection of the neutrophils from a sample obtained from a subject will allow a skilled person to diagnose a particular disease state and then seek to treat it. For example, the presence of neutrophils may indicate inflammation, cancer, a transplanted organ in danger of rejection and a wound in need to intervention (i.e. wound healing). As such, the skilled person may instigate therapies for the treatment of inflammation, cancer, organ rejection and wound healing, respectively. As will be appreciated, the steps above may be conducted on an analyte obtained from a subject, where the analyte is subjected to an in vitro test.

For the avoidance of doubt, in the context of the present invention, the term “treatment” includes references to therapeutic or palliative treatment of patients in need of such treatment, as well as to the prophylactic treatment and/or diagnosis of patients who are susceptible to the relevant disease states.

In addition to the use case of the compounds of formula I, or it salts and solvates, in vitro, it may also be used in vivo. Thus, in a further aspect of the invention, there is provided a method for detection of neutrophil elastase in vivo, the method comprising the following steps:
(ai) administering a compound of formula I, or pharmaceutically acceptable salt or solvate thereof, as described hereinbefore to a subject; and
(aii) detecting any chemiluminescence, wherein the presence of neutrophil elastase in vivo is indicated by chemiluminescence.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The administration methods used herein may, for example, include skin-delivery with the assistance of microneedle, intraperitoneal injection, and intravenous injection. For example, the administration may make use of a poly(methyl methacrylate) microneedle.

The same diseases mentioned hereinbefore may also be treated following diagnosis in vivo.

In a further aspect of the invention, there is provided a use of a compound of formula I or a salt and/or solvate thereof as described herein for the manufacture of a diagnostic agent for in vivo diagnosis of a disease caused by neutrophil elastase proliferation.

In a yet further aspect, there is provided a compound of formula I or a salt and/or solvate thereof as described herein for use in the in vivo diagnosis of a disease caused neutrophil elastase proliferation.

In a still aspect of the invention, there is provided a method of diagnosis of a disease caused neutrophil elastase proliferation, involving administering to a subject in need thereof a composition comprising a compound of formula I or a salt and/or solvate thereof as described herein and detecting a signal, the detection of which indicates a disease caused neutrophil elastase proliferation in said subject.

Again, the diseases that may be detected by neutrophil elastase proliferation are described hereinbefore. As will be appreciated, the skilled person making the diagnosis may then treat the subject according to the presence (or absence) of the disease in question.

The compounds of formula I may be useful in identifying therapeutic compounds for the treatment of a number of diseases, such as psoriasis and peritonitis. Thus, in a further aspect of the invention, there is provided a method for identifying a compound suitable for the treatment of psoriasis, the method comprising:
(bi) providing a mouse where the skin exhibits neutrophil infiltration (e.g. where the dorsal skin of the mouse has been treated by imiquimod);
(bii) contacting the skin with a test material for a first period of time;
(biii) after the first period of time, contacting the drug-treated skin with a compound of formula I, or a pharmaceutically acceptable salt or solvate thereof, as defined herein for a second period of time; and
(biv) after the second period of time any chemiluminescent signal skin is detected and compared to a blank control, where a reduced quantitative chemiluminescence readout in the presence of the test compound compared to the blank control is indicative of anti-psoriasis activity.

In a further aspect of the invention, there is provided a method for identifying a compound suitable for the treatment of peritonitis, the method comprising:
(ci) providing a mouse suffering from peritonitis, where the peritonitis is associated with ascites, where the ascites exhibits neutrophil infiltration (e.g where the mouse's peritonitis was induced by intraperitoneal injection of lipopolysaccharides (LPS));
(cii) contacting an abdomen of the mouse with a test material for a first period of time;
(ciii) after the first period of time, contacting the LPS-treated abdomen with a compound of formula I, or a pharmaceutically acceptable salt or solvate thereof, as defined herein for a second period of time; and
(civ) after the second period of time any chemiluminescent signal abdomen is detected and compared to a blank control, where a reduced quantitative chemiluminescence readout in the presence of the test compound compared to the blank control is indicative of peritonitis activity,
where mice were treated with PBS only.

The aspects of the invention described herein (e.g. the above-mentioned compounds, combinations, methods and uses) may have the advantage that, in the diagnosis of the conditions described herein, they may be more convenient for the physician and/or patient than, be more efficacious than, be less toxic than, have better selectivity over, be more selective than, be more sensitive than, produce fewer side effects than, or may have other useful pharmacological properties over, similar compounds, combinations, methods (treatments) or uses known in the prior art for use in the diagnosis of those conditions or otherwise.

Further aspects and embodiments of the invention will be described by reference to the following non-limiting examples.

### EXAMPLES

### Materials

All the mentioned chemicals containing anhydrous solvents which were used in our synthesis were purchased from Sigma-Aldrich and TCI companies without further purification. For example, ethylenediaminetetraacetic acid (EDTA), PBS, tetra-n-butylammonium fluoride (TBAF), (4-(2-hydroxyethyl)-1-piperazineethanesulfonic acid) (HEPES), and cyclosporin A (CsA), were purchased from Sigma-Aldrich. NE, other enzymes (APN, ALP, Cas-3, Cat B, GGT, furine, β-gal, CatG, PR3, NTR, neutrophil antibodies (CD11c+ and APC-labeled Ly6G+) and LPS were purchased from Sigma-Aldrich Co., Ltd and R&D systems, inc. companies. The cell RPMI 1640 Medium, fetal bovine serum (FBS), streptomycin, and penicillin were purchased from Thermo Fisher Scientific Co., Ltd. MTS assay was supplied by Cell Signaling Technology Company. Both the NaOCl and H₂O₂ solutions were commercial products and purchased from Sigma-Aldrich. Aquaphor was ordered from Watsons Singapore. Poly(methyl methacrylate) microneedle was prepared by microneedle template supplied from Micropoint Technologies Pte Ltd.

### Analytical Techniques

### ¹H NMR Spectroscopy

¹H NMR spectra were recorded by using a Bruker BBFO 400 MHz NMR.

### Liquid Chromatography-Mass Spectroscopy (LC-MS)

LC-MS analyses were tested with Triple Quadrupole LC/MS (Agilent 1260-6460).

### HPLC

HPLC analyses were carried out on an Agilent 1260 system using methanol/water as the eluent.

### Ultraviolet-Visible (UV-Vis) Spectroscopy

The UV-vis spectra were measured on a Shimadzu UV-2450 spectrophotometer.

### Fluorescence Spectroscopy

The fluorescence spectra and QY were tested on a Fluorolog 3-TCSPC spectrofluorometer or SpectraMax M.

### Fluorescence and Chemiluminescence

Fluorescence and chemiluminescence of the chemiluminescent probes were tested by using IVIS spectrum imaging system and a microplate reader, respectively.

### Statistical Analysis

The in vivo chemiluminescence intensities were carried on ROI analysis using Living Image 4.0 Software. All in vitro and in vivo data were expressed as the mean±standard deviation unless otherwise stated. The experiment mice should be blindly and randomly divided to three groups with 3 mice per group. Statistical comparisons between the two groups were determined by Student's t-test (2-tailed, unpaired) and P values (p*<0.05, P**<0.01, P***<0.001) were considered as statistically significant meaning. The intensity of cell imaging was performed using ImageJ.

### Example 1. Synthesis of Chemiluminescent Probes (BOPD_{Su}, BTPD_{Su}, MBPD_{Su}, MPD_{Su}, BTPD_{Ne}, MPD_{Ne} and BTPD)

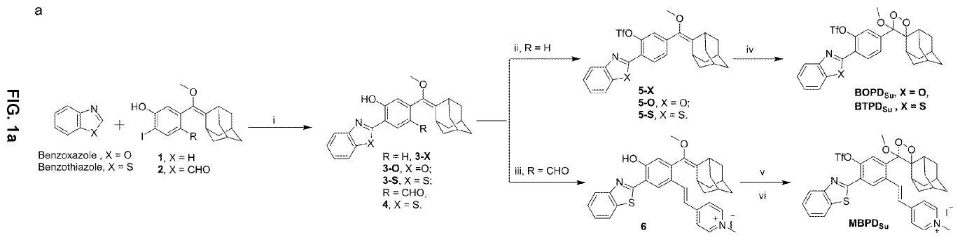

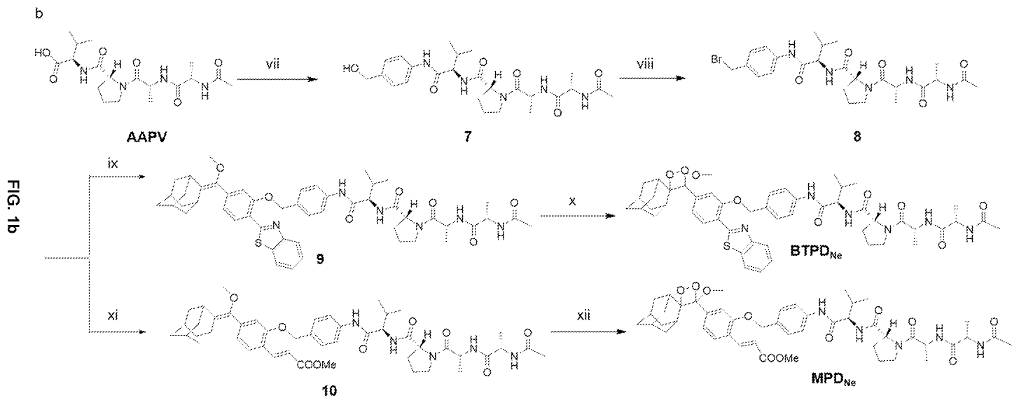

We herein report the synthesis of ultrabright activatable chemiluminescent probes with intramolecular H-bonding-prolonged half-lives for in vivo imaging of neutrophils. As shown in FIG. 1a, the chemiluminescence half-life of phenoxyl-dioxetane substrate is affected by the equilibrium between deprotonation and protonation of phenolic hydroxyl, while the chemiluminescence brightness is determined by benzoate ester product after activation. Thus, it was envisioned that by introducing benzoazole derivatives onto the ortho position of phenol in adamantylidene-1,2-dioxetane to induce the intramolecular H-bond of phenol-dioxetane intermediate, the deprotonation of phenolic hydroxyl intermediate can be delayed (FIG. 1b), ultimately prolonging the chemiluminescence. In addition, because of the high fluorescence QYs of benzoazole derivatives, it is expected that the chemiluminescence QYs of such substrates can be increased.

To study the chemiluminescence properties of benzoazole-phenoxyl-dioxetane substrates, three model activatable probes (termed as benzoxazole-phenoxyl-dioxetane (BOPD_{Su}), benzothiazole-phenoxyl-dioxetane (BTPD_{Su}) and 1-methylpyridinium-benzothiazole-phenoxyl-dioxetane (MBPD_{Su}) were synthesized to respond to superoxide (O₂^{•-}). Firstly, compounds 3-X (X=O and S) and 4 were obtained by C-H/C-I cross-coupling reaction between corresponding azoles (benzothiazole or benzoxazole) and aryl iodide derivatives (1 or 2). Knoevenagel condensation between O₂^{•-} and 1,4-dimethylpyridinium iodide led to 6. Then, 3-X and 6 were caged with O₂^{•-}-responsive trifluoromethanesulfonate (T_{f}) group to yield 5-X and 7, respectively (Hu, J. J. et al., J. Am. Chem. Soc. 2015, 137, 6837-6843). Finally, BOPD_{Su}, BTPD_{Su} and MBPD_{Su} were synthesized by oxidation of compounds 5-X and 7 with singlet oxygen (¹O₂), respectively. The O₂^{•-}-responsive control probe (MPDₛᵤ) was also synthesized for comparison.

### Compound 3-X (R=H, X=O or S)

The mixture of azole derivative (0.3 mmol), compound 1 (0.3 mmol), Pd(OAc)₂ (3.6 mg, 0.015 mmol), PPh₃ (4.0 mg, 0.03 mmol), CuI (11.4 mg, 0.06 mmol), and Cs₂CO₃ (195 mg, 0.6 mmol) in dimethylacetamide (2.5 mL) were reacted for 6 h at 145° C. under nitrogen atmosphere.

After the reaction, the mixture was cooled, filtered, and extracted with saline and DCM for three times. The organic solvent was collected, dried over anhydrous Na₂SO₄, and evaporated under a vacuum. The crude product was purified by silica gel chromatography using hexane/ethyl acetate as an eluent to give the corresponding product.

3-O (white solid, 44.1 mg, 38% yield). ¹H NMR (CDCl₃, 400 MHz): δ 11.46 (s, 1H), 8.00 (d, J=8 Hz, 1H), 7.23-7.75 (m, 1H), 7.61-7.63 (m, 1H), 7.38-7.40 (m, 2H), 7.09 (d, J=1.2 Hz, 1H), 7.01 (dd, J₁=1.2 Hz, J₂=1.6 Hz, 1H), 3.36 (s, 3H), 3.28 (s, 1H), 2.77 (s, 1H), 1.84-2.00 (m, 13H). MS (ESI-): m/z=386.2 [M−H]⁻.

3-S (white solid, 37.5 mg, 31% yield). ¹H NMR (CDCl₃, 400 MHz): δ 12.50 (s, 1H), 8.00 (d, J=8 Hz, 1H), 7.91 (d, J=7.6 Hz, 1H), 7.68 (d, J=8 Hz, 1H), 7.49-7.54 (m, 1H), 7.39-7.43 (m, 1H), 7.07 (d, J=1.2 Hz, 1H), 6.96 (dd, J, =1.6 Hz, J₂=1.6 Hz, 1H), 3.36 (s, 3H), 3.28 (s, 1H), 2.78 (s, 1H), 1.84-2.00 (m, 13H). MS (ESI-): m/z=402.2 [M−H]⁻.

### Compound 4 (R=CHO, X=S).

Compound 4 was prepared from compound 2 by following the synthesis method for compound 3-X. 4 (white solid, 53 mg, 41% yield). ¹H NMR (CDCl₃, 400 MHz): δ 13.29 (s, 1H), 10.16 (s, 1H), 8.39 (s, 1H), 8.02 (d, J=8 Hz, 1H), 7.96 (d, J=7.6 Hz, 1H), 7.53-7.57 (m, 1H), 7.45-7.49 (m, 1H), 7.03 (s, 1H), 3.36 (s, 3H), 3.34 (s, 1H), 2.38 (s, 1H), 1.73-2.00 (m, 12H). MS (ESI-): m/z=430.1 [M−H]⁻.

### Compound 5-X (R=H, X=O or S)

Trifluoromethanesulfonic anhydride (Tf₂O, 1 mmol, 168 μL) was added dropwise to a solution of compound 3-X (0.5 mmol) in pyridine and DCM (1/10, v/v, total 3 mL) under nitrogen atmosphere. Then, the reaction was stirred for 3 h and the mixture was extracted with saline and DCM for three times. The organic solvent was collected, dried over anhydrous Na₂SO₄, and evaporated under vacuum. The crude product was purified by silica gel chromatography using hexane/ethyl acetate as an eluent to give a white solid.

5-O (white solid, 24.6 mg, 95% yield). ¹H NMR (CDCl₃, 400 MHz): δ 8.36 (d, J=8.4 Hz, 1H), 7.83-7.85 (m, 1H), 7.62-7.64 (m, 1H), 7.51 (dd, J, =1.6 Hz, J₂=1.2 Hz, 1H), 7.40-7.43 (m, 3H), 3.36 (s, 3H), 3.29 (s, 1H), 2.74 (s, 1H), 1.80-2.01 (m, 12H). MS (ESI⁺): m/z=520.2 [M+H]+.

5-S (white solid, 24.3 mg, 91% yield). ¹H NMR (CDCl₃, 400 MHz): δ 8.16 (d, J=8 Hz, 1H), 8.11 (d, J=8 Hz, 1H), 7.96 (d, J=7.6 Hz, 1H), 7.53-7.57 (m, 1H), 7.44-7.50 (m, 3H), 3.37 (s, 3H), 3.29 (s, 1H), 2.74 (s, 1H), 1.80-2.01 (m, 13H). MS (ESI⁺): m/z=536.2 [M+H]+.

### Compound 6

To a solution of compound 4 (25 mg, 0.06 mmol) and 1,4-dimethylpyridinium iodide (18.8 mg, 0.08 mmol) in ACN (3 mL) was added 20 μL of piperidine under N₂ atmosphere. Then, the mixture was refluxed for 2 h. After finishing the reaction, the mixture was extracted with saline and DCM for three times. The organic solvent was collected, dried over anhydrous Na₂SO₄, and evaporated under vacuum. The crude product was purified by silica gel chromatography using DCM/methanol as an eluent to give compound 6 (pale yellow solid, 26.4 mg, 68% yield).

¹H NMR (MeOD, 400 MHz): δ 8.70 (d, J=6.8 Hz, 1H), 8.55 (s, 1H), 8.06-8.09 (m, 4H), 7.98 (d, J=16.4 Hz, 1H), 7.57-7.61 (m, 1H), 7.44-7.52 (m, 2H), 7.33-7.35 (m, 1H), 7.02 (s, 1H), 4.30 (s, 3H), 3.36 (s, 4H), 2.26 (s, 1H), 1.79-2.01 (m, 13H). MS (ESI⁺): m/z=520.2 [M-I]⁺.

The General Method to Synthesize BOPD_{Su} and BTPD_{Su}

A mixture of compound 5-X (0.04 mmol) and a catalytic amount of methylene blue (4 mg) was dissolved in DCM (10 mL). Air was bubbled through the solution while irradiating with white light (LED 150 W) at 0° C. for 6 h. The organic solvent was then removed by rotary evaporator under vacuum and the crude product was purified by HPLC to give the final pure product.

BOPD_{Su} (white solid, 20 mg, 91%). ¹H NMR (CDCl₃, 400 MHz): δ 8.49 (d, J=8.4 Hz, 1H), 8.21 (dd, J, =1.6 Hz, J₂=1.2 Hz, 1H), 8.10 (m, 1H), 7.85-7.88 (m, 1H), 7.64-7.66 (m, 1H), 7.41-7.48 (m, 1H), 4.00 (s, 1H), 1.49-1.67 (m, 11H), 0.92-1.01 (m, 2H). MS (ESI⁺): m/z=552.3 [M+H]+.

BTPD_{Su} (white solid, 21.5 mg, 95%). ¹H NMR (CDCl₃, 400 MHz): δ 8.27 (d, J=8.4 Hz, 1H), 8.16-8.20 (m, 2H), 8.13 (d, J=1.2 Hz, 1H), 7.98 (d, J=8 Hz, 1H), 7.55-7.59 (m, 1H), 7.47-7.51 (m, 1H), 7.33-7.35 (m, 1H), 7.02 (s, 1H), 4.00 (s, 3H), 3.28 (s, 1H), 1.61-2.13 (m, 11H), 1.28-1.42 (m, 2H). MS (ESI⁺): m/z=568.2 [M+H]+.

### MBPD_{Su}

Firstly, to a solution of compound 6 (20 mg, 0.03 mmol) and pyridine (200 μL) in dry DCM (2 mL), Tf₂O (0.1 mmol, 13 μL) was added at 0° C. under N₂ atmosphere. Then, the reaction was stirred for 3 h and the mixture was extracted with saline and DCM for three times. The organic solvent was collected, dried over anhydrous Na₂SO₄, and evaporated under vacuum. The crude product was not further purified. Secondly, the crude product and catalytic amount of methylene blue (4 mg) was dissolved in DCM (10 mL). Air was bubbled through the solution while irradiating with white light (LED 150 W) at 0° C. for 6 h. The organic solvent was then removed by rotary evaporator under vacuum and the crude product was purified by HPLC to give MBPD_{Su} (yellow solid, 21.6 mg, 92%).

¹H NMR (MeOD, 400 MHz): δ 8.75 (d, J=6.8 Hz, 1H), 8.71 (s, 1H), 8.64 (d, J=16.4 Hz, 1H), 8.18 (d, J=16.4 Hz, 1H), 8.05-8.09 (m, 2H), 7.96 (s, 1H), 7.55-7.57 (m, 1H), 7.47-7.51 (m, 2H), 7.37-7.40 (m, 1H), 7.01 (s, 1H), 4.34 (s, 3H), 4.00 (s, 3H), 1.55-1.75 (m, 11H), 0.95-1.00 (m, 2H). MS (ESI⁺): m/z=685.2 [M-I]+.

### Compound 7

To solution of peptide AAPV (398.2 mg, 1 mmol) and p-aminobenzyl alcohol (246.4 mg, 2 mmol) in DCM (10 ml), EEDQ (495 mg, 2 mmol) was added under N₂ atmosphere. The mixture was stirred for 6 h at room temperature. Upon complete reaction, the organic solvent was removed under vacuum and the mixture was purified by HPLC to give compound 7 (yellow solid, 437.8 mg, 87%).

7. ¹H NMR (MeOD, 400 MHz): δ 7.49-7.58 (m, 2H), 7.29-7.39 (m, 1H), 4.50-4.64 (m, 4H), 4.31-4.39 (m, 1H), 4.26 (d, J=7.2 Hz, 1H), 3.76-3.86 (m, 1H), 3.60-3.68 (m, 1H), 2.11-2.37 (m, 2H), 1.97-2.22 (m, 3H), 1.96 (s, 3H), 1.28-1.38 (m, 6H), 0.97-1.07 (m, 6H). MS (ESI⁺): m/z=503.2 [M]⁺.

Peptide AAPV. AAPV was synthesized with solid-phase peptide synthesis (SPPS). ¹H NMR (MeOD, 400 MHz): δ 4.61 (m, 1H), 4.55 (dd, J₁=J₂=3.6 Hz, 1H), 4.31-4.36 (m, 1H), 4.27-4.30 (m, 1H), 3.76-3.82 (m, 1H), 3.63-3.68 (m, 1H), 2.11-2.25 (m, 2H), 1.99-2.11 (m, 3H), 1.97 (s, 3H), 1.31-1.36 (m, 6H), 0.95-1.02 (m, 6H). MS (ESI⁺): m/z=398.2 [M]⁺.

### Compound 9

To a solution of compound 7 (151 mg, 0.3 mmol) in THF (5 ml), PBr₃ (85 μL, 0.9 mmol) was added at 0° C. under N₂ atmosphere and the reaction mixture was stirred for 3 h. Upon complete reaction, the reaction mixture was quenched with saturated NaHCO₃, extracted with saline and ethyl acetate. The organic was collected and dried with anhydrous Na₂SO₄ and removed by vacuo to give crude product (compound 8) without further purification.

The crude product 8 was added into a solution of 3-S (60.5 mg, 0.15 mmol) and N,N-diisopropylethylamine (52 μL, 0.3 mmol) in ACN (3 mL) under N₂ atmosphere. The mixture was stirred at 60° C. overnight. After complete reaction, the organic solvent was removed by vacuo and the mixture was purified by HPLC to give compound 9 (yellow solid, 30.7 mg, 23%).

¹H NMR (MeOD, 400 MHz): δ 8.42 (m, 1H), 8.02 (m, 1H), 7.97 (m, 1H), 7.71-7.73 (m, 1H), 7.60-7.63 (m, 2H), 7.42-7.52 (m, 2H), 7.39-7.43 (m, 1H), 7.06-7.11 (m, 2H), 5.42 (s, 2H), 4.54-4.63 (m, 4H), 4.32-4.35 (m, 2H), 3.58-3.61 (m, 1H), 3.47-3.52 (m, 1H), 3.28 (s, 3H), 1.98-2.21 (m, 8H), 1.78-1.89 (m, 13H), 1.34-1.48 (m, 6H), 0.99-1.04 (m, 6H). MS (ESI⁺): m/z=889.1 [M]⁺.

BTPD_{Ne} and its classical counterpart MPD_{Ne}, were synthesized by caging the corresponding chemiluminophores with a NE-cleavable peptide (AAPV) (FIG. 1b).

### BTPD_{Ne}

A mixture of compound 9 (26.7 mg, 0.03 mmol) and catalytic amount of methylene blue (7 mg) was dissolved in DCM (30 mL). Air was bubbled through the solution while irradiating with white light (LED 150 W) at 0° C. for 6 h. The organic solvent was then removed by rotary evaporator under vacuum and the crude product was purified by HPLC to give BTPD_{Ne} (yellow solid, 26.5 mg, 96%).

¹H NMR (CDCl₃, 400 MHz): δ 8.62 (m, 1H), 8.12 (d, J=6.8 Hz, 1H), 7.99 (d, J=7.6 Hz, 1H), 7.52-7.79 (m, 2H), 7.29-7.65 (m, 5H), 6.99-7.00 (m, 1H), 5.34 (s, 2H), 4.07-4.35 (m, 6H), 3.96 (s, 3H), 3.51 (m, 1H), 2.17-2.41 (m, 8H), 1.85-2.10 (m, 11H), 1.43-1.51 (m, 6H), 1.07-1.12 (m, 2H), 0.93-1.12 (m, 6H). MS (ESI⁺): m/z=920.3 [M]⁺.

### MPD_{Ne}

Compound 10 (without further purification) by following the synthesis protocol for compound 9. Compound 10 was verified by LC-MS (m/z=840.25 [M+H]⁺) and the yield was determined by HPLC. MPD_{Ne} was prepared from crude compound 10 (8.4 mg, 0.01 mmol) by following the synthesis protocol for BTPD_{Ne} except methylene blue (1 mg) was used.

MPD_{Ne} (white solid, 3.6 mg, 41% for two steps yield). ¹H NMR (CDCl₃, 400 MHz): δ 8.36 (s, 1H), 8.02 (d, J=16.4 Hz, 1H), 7.61 (d, J=8.4 Hz, 1H), 7.45-7.52 (m, 1H), 7.33-7.38 (m, 2H), 6.89-6.93 (m, 2H), 6.53 (d, J=16 Hz, 1H), 5.10 (s, 2H), 4.75-4.82 (m, 1H), 4.75-4.82 (m, 1H), 4.64-4.68 (m, 2H), 4.34 (t, J=5.8 Hz, 1H), 4.06-4.2 (m, 2H), 3.74-3.81 (s, 3H), 3.64-3.66 (m, 1H), 3.26 (s, 3H), 2.05-2.43 (m, 8H), 1.75-1.98 (m, 11H), 1.42-1.28 (m, 6H), 0.86-1.01 (m, 6H). MS (ESI⁺): m/z=871.3 [M+H]⁺.

### BTPD

BTPD was prepared from 3-S (20.2 mg, 0.05 mmol) by following the synthesis protocol for BTPD_{Ne} except methylene blue (5 mg) was used.

BTPD (white solid, 20.4 mg, 94%). ¹H NMR (CDCl₃, 400 MHz): δ 12.60 (s, 1H), 8.03 (d, J=8.0 Hz, 1H), 7.94 (d, J=8.0 Hz, 1H), 7.75-7.77 (m, 2H), 7.62 (d, J=8.4 Hz, 1H), 7.55 (t, J=7.4 Hz, 1H), 7.46 (t, J=7.4 Hz, 1H), 3.95 (s, 3H), 3.28 (s, 1H), 1.43-2.26 (m, 11H), 1.25-1.31 (m, 2H). MS (ESI⁺): m/z=436.2 [M+H]⁺.

### Example 2. Synthesis of Activated Probes (ABOPD, ABTPD and AMBPD)

FIG. 1c depicts the synthesis routes of the activated probes.

### ABOPD and ABTPD

ABOPD (activated BOPD_{Su}) and ABTPD (activated BTPD_{Su}) were prepared by following the synthesis protocol for compound 3-X in Example 1.

ABOPD (pale yellow solid, 46.8 mg, 58% yield). ¹H NMR (CDCl₃, 400 MHz): δ 8.11 (d, J=8.4 Hz, 1H), 7.69-7.79 (m, 2H), 7.63-7.68 (m, 2H), 7.42-7.64 (m, 2H), 3.36 (s, 3H). MS (ESI-): m/z=268.1 [M−H]⁻.

ABTPD (pale yellow solid, 38.4 mg, 45% yield). ¹H NMR (DMSO-de, 400 MHz): δ 11.81 (s, 1H), 8.42 (d, J=6.4 Hz, 1H), 8.17 (d, J=6.4 Hz, 1H), 8.10 (d, J=6.4 Hz, 1H), 7.67 (d, J=1.2 Hz, 1H), 7.55-7.60 (m, 2H), 7.46-7.49 (m, 1H), 3.36 (s, 3H). MS (ESI-): m/z=284.1 [M−H]⁻.

### AMBPD (Activated MBPD_{Su})

A mixture of compound 6 (0.1 mmol) and catalytic amount of methylene blue (6 mg) was dissolved in DCM (20 mL). Air was bubbled through the solution while irradiating with white light (LED 150W) for 6 h. Upon reaction completion, the organic solvent was removed by vacuo to give crude product without further purification. The crude product was dissolved in methanol (20 mL). The K₂CO₃ (2 mmol) was added in the solution. The reaction mixture was stirred for 2 h. Upon complete reaction, the organic solvent was removed by vacuo and washed with brine (20 mL) and DCM (50 mL) for three times. Then, the organic layer was dried with anhydrous Na₂SO₄, filtered and concentrated. The crude product was purified by HPLC to give AMBPD (yellow solid, 35.5 mg, 67%).

¹H NMR (MeOD, 400 MHz): δ 8.72 (d, J=6.0 Hz, 1H), 8.55 (m, 2H), 8.11 (dd, J, =5.2 Hz, J₂=7.2 Hz, 4H), 7.66 (s, 1H), 7.60 (t, J=11.8 Hz, 1H), 7.51 (t, J=7 Hz, 1H), 7.36 (d, J=16.8 Hz, 1H), 4.33 (s, 3H), 3.97 (s, 3H). MS (ESI⁺): m/z=403.1 [M-I]⁺.

### Example 3. Preparation of Different ROS Solutions

KO₂ (8 mg) was dissolved in anhydrous DMSO solution (16 mL) as the stock solution for the following tests. Hydroxyl radical (•OH) was produced by Fenton reaction between H₂O₂ and FeSO₄·7H₂O; singlet oxygen (¹O₂) was obtained by adding NaOCl to H₂O₂; and sodium peroxynitrite was synthesized by mixing acidified H₂O₂ with NaNO₂ in NaOH solution, then concentration of peroxynitrite anion was determined by UV absorption at 302 nm. Solutions of ROS (1.0 mM), MgSO₄ (1.0 mM), CaCl₂ (1.0 mM), and FeSO₄ (1.0 mM) were well-prepared before test.

### Example 4. Optical Properties and Sensing Abilities of Chemiluminescent Probes (BOPD_{Su}, BTPD_{Su}, MBPD_{Su}, MPD_{Su}, BTPD_{Ne} and MPD_{Ne})

To study the optical properties and sensing abilities of BOPD_{Su}, BTPD_{Su} and MBPD_{Su} toward O₂^{•-}, their absorption, chemiluminescence and fluorescence spectra were measured.

Selectivity of BOPD_{Su}, BTPD_{Su}, MBPD_{Su} and MPD_{Su}

The ROS solutions were prepared in Example 3. All the mentioned ion solutions MgSO₄ (1.0 mM), CaCl₂ (1.0 mM), FeSO₄·7H₂O (1.0 mM), were prepared before the tests.

The chemiluminescence and fluorescence changes of BOPD_{Su}, BTPD_{Su}, MBPD_{Su} and MPD_{Su} (20 μM) were recorded at different concentrations of RONS (40 μM) and other metal ions (100 μM). BOPD_{Su}, BTPD_{Su}, MBPD_{Su} and MPD_{Su} (20 μM) in phosphate buffered saline (PBS) buffer were treated in the presence of different RONS (40 μM), and metal ions (Mg²⁺, Ca²⁺ or Fe²⁺, 100 μM) at 37° C.

Chemiluminescence and fluorescence changes of BTPD_{Ne} (20 μM) were determined in the presence of NE (0.1 U/ml), other different enzymes (˜0.1 U/mL, APN, ALP, Cas-3, cat B, GGT, β-gal and NTR) in 50 mM Tris, 1 M NaCl, 0.05% (w/v) Brij-35, pH 7.5 at 37° C. after 30 min incubation.

The chemiluminescence signals were recorded at different concentrations of KO₂ (0, 50, 100, 250, 500 and 1000 nM). Then, the limit of detection (LOD) of BOPD_{Su}, BTPD_{Su}, MBPD_{Su} and MPD_{Su} were calculated by chemiluminescence intensities based on the equation: LOD=3σ/k, where σ is the standard deviation of emission intensity of blank, and k is the slope of plot of emission intensities.

To perform chemiluminescence kinetic studies, chemiluminescence intensities of BOPD_{Su}, BTPD_{Su}, MBPD_{Su} and MPD_{Su} (20 μM) were acquired in the presence of KO₂ (40 μM), respectively. The chemiluminescent intensities were plotted as a function of time.

The chemiluminescence QYs of BOPD_{Su}, BTPD_{Su}, MBPD_{Su}, and MPD_{Su} (20 μM) were measured in the presence of O₂^{•-} (100 μM) in PBS (10 mM, pH 7.4, 10% DMSO) at 37° C. The fluorescence QYs were determined by using Rhodamine B as a standard dye (QY 36%) in PBS (10 mM, pH 7.4) (X. Zhen et al., ACS Nano 2016, 10, 6400-6409). The fluorescence QY of AMPD was obtained from the literature (O. Green et al., ACS Cent. Sci. 2017, 3, 349-358).

Selectivity of BTPD_{Ne} and MPD_{Ne}

Chemiluminescence and fluorescence changes of BTPD_{Ne} and MPD_{Ne} (20 μM) in the presence of NE (0.1 U/mL), or other different enzymes (˜0.1 U/mL, APN, ALP, Cas-3, Cat B, GGT, β-gal, NTR, CatG and PR3) were tested after 30 min incubation in 50 mM Tris, 1 M NaCl, 0.05% (w/v) Brij-35, pH 7.5 at 37° C.

To determine the LOD for NE enzyme detection, the chemiluminescence of BTPD_{Ne} was recorded in 50 mM Tris, 1 M NaCl, 0.05% (w/v) Brij-35, pH 7.5 at 37° C. with different concentrations of NE (0, 0.05, 0.1, 0.5, 1, and 2 U/ml) for 30 min incubation. Then, initial reaction velocity was calculated.

To carry out the kinetic assay, various concentrations of BTPD_{Ne} (2, 5, 10, 20, 40 and 80 μM) were incubated with NE (0.1 U/mL) at 37° C. for 10 min in 50 mM Tris, 1 M NaCl, 0.05% (w/v) Brij-35, and at pH=7.5. After incubation, quantification analyses were determined by HPLC. The initial reaction velocity was calculated, plotted against the concentration of BTPD_{Ne}, and fitted to a Michaelis-Menten curve. Finally, the kinetic parameters were calculated by Michaelis Menten equation:

$$v = \frac{{v}_{max} [ S ]}{{K}_{m} + [ S ]} ,$$

where v is initial velocity, and [S] is substrate concentration.

### Results and Discussion

BOPD_{Su}, BTPD_{Su} and MBPD_{Su} showed respective maximum absorption at 308, 320 and 344 nm, negligible chemiluminescence and very low fluorescence in the absence of O₂^{•-}. This is because the electron-donating ability of phenol in adamantylidene-1,2-dioxetane is diminished at “caged” state.

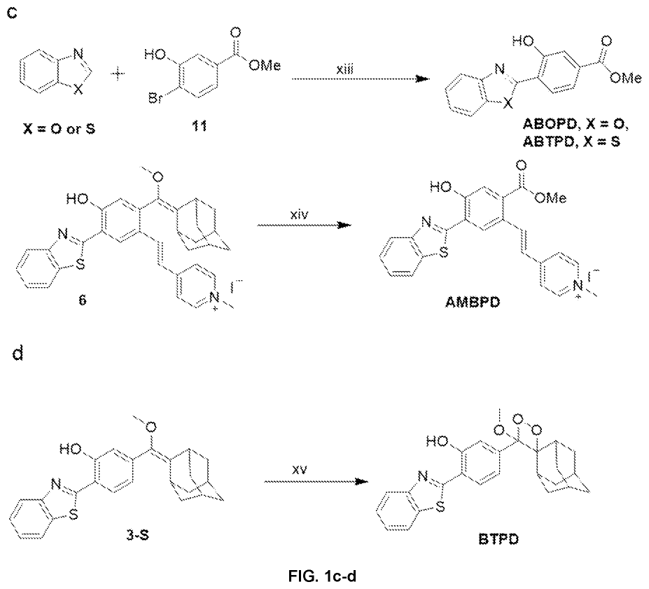

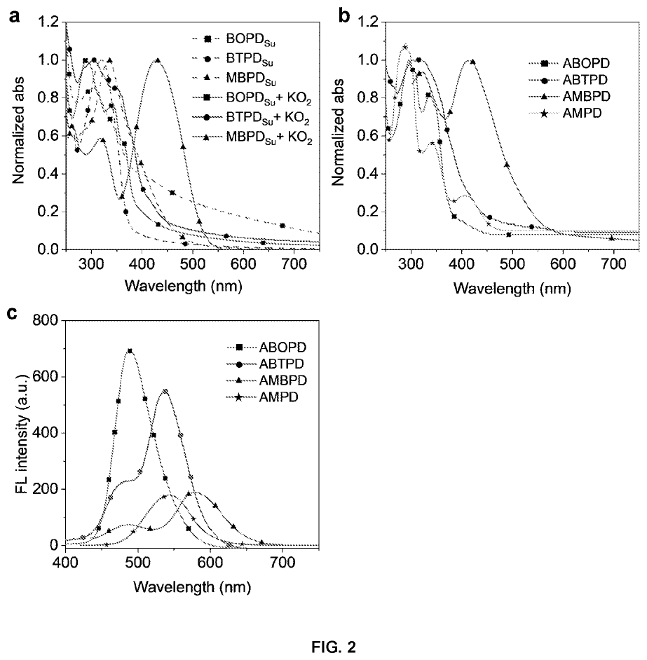

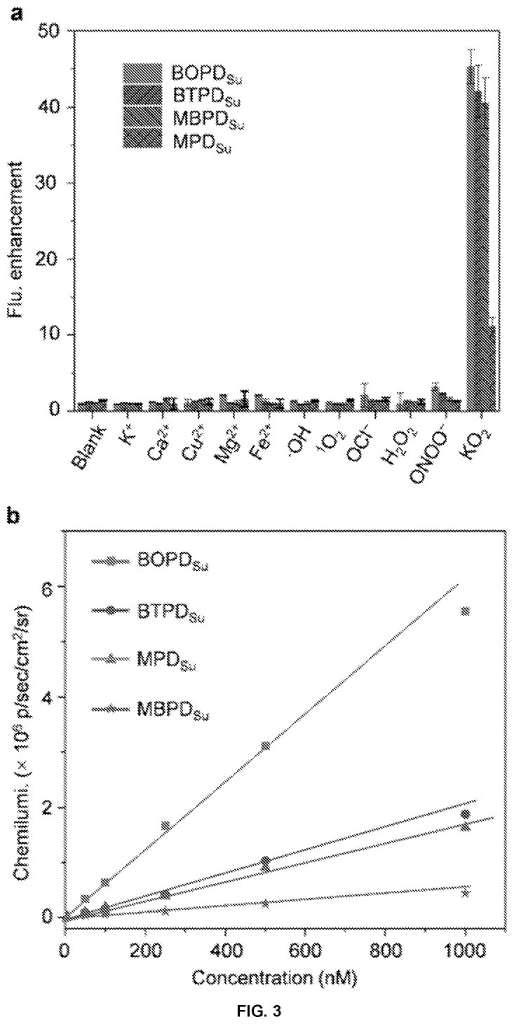

However, upon cleavage of sulfonate ester group by O₂^{•-}, BOPD_{Su}, BTPD_{Su} and MBPD_{Su} (FIG. 2) exhibited new absorption peaks at 342, 344 and 428 nm and fluorescent signal enhancements by 45.3 (at ˜486 nm), 42.1 (at 532 nm), and 40.5 folds (at 582 nm), respectively. The fluorescence enhancement of our designed probes was ˜3.6-4.0-fold higher than that of MPD_{Su} (11.2 times at 542 nm, FIG. 3), which was attributed to the higher fluorescence intensities of the benzoate esters with the excited state intramolecular proton transfer (ESIPT, Sedgwick, A. C. et al., Chem. Soc. Rev. 2018, 47, 8842-8880).

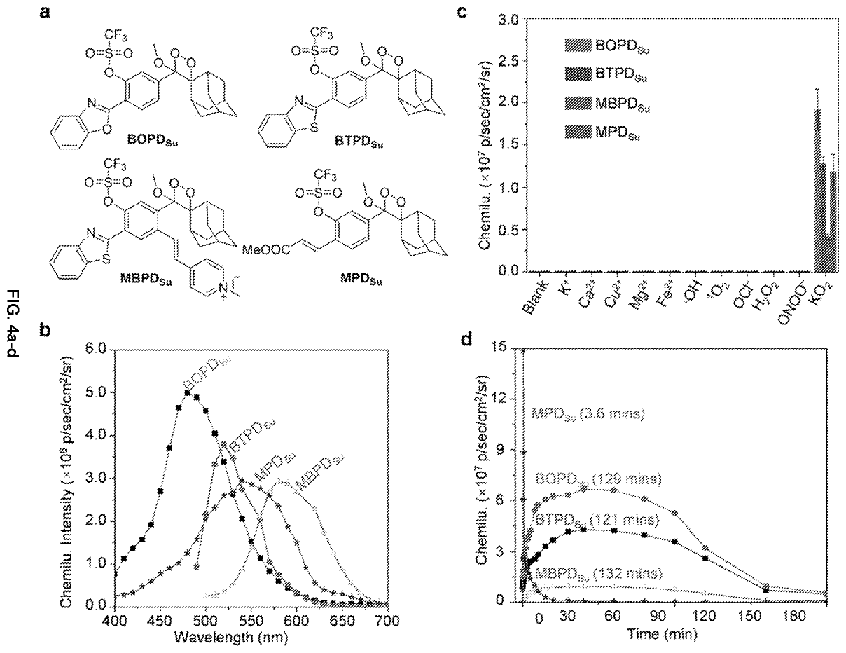

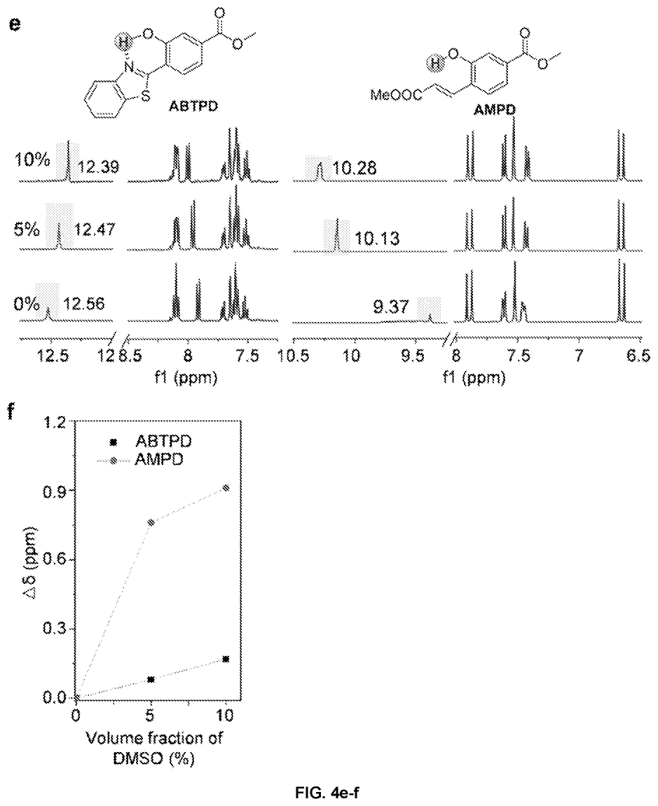

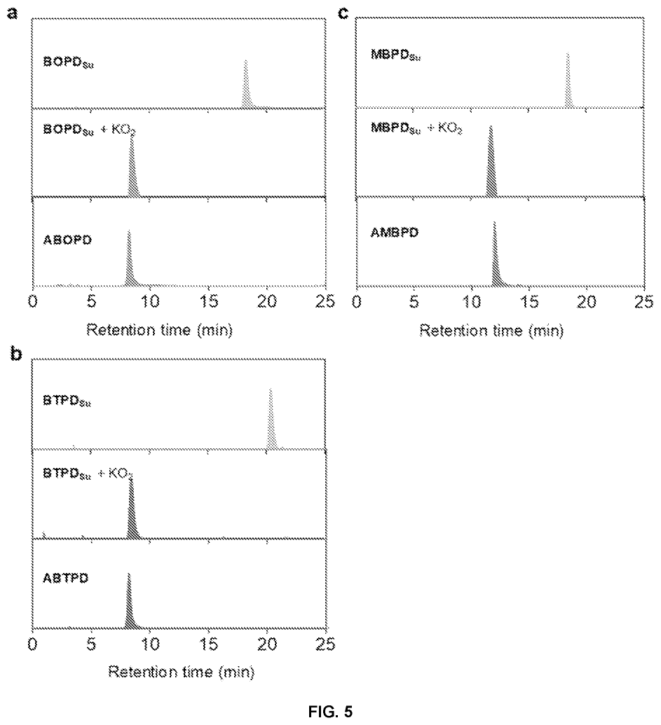

The chemiluminescence spectra of BOPD_{Su}, BTPD_{Su}, MBPD_{Su} and MPD_{Su} were similar to their corresponding fluorescence spectra (FIGS. 2 and 4b). The chemiluminescence intensities of BOPD_{Su}, BTPD_{Su}, MBPD_{Su} and MPD_{Su} were increased respectively by 3291 times (at 480 nm), 2960 times (at 520 nm), 2590 times (at 580 nm), and 2624 times (at 540 nm) after addition of O₂^{•-} for 10 s, which were ascribed to the formation of the corresponding unstable phenolate dioxetane intermediate after deprotection of sulfonate ester group by O₂^{•-} (Huang, J. et al., Nat. Mater. 2019, 18, 1133-1143). However, chemiluminescence signals of these probes toward other ROS and metal ions showed no obvious changes, and the high selectivity was confirmed by the weak chemiluminescence of these probes toward other ROS and metal ions (FIG. 4c). The BAPD-based probes had similar LODs against O₂^{•-} at ˜5.7 nM, 6.8 nM and 6.3 nM which are slightly lower than the LOD of MPD_{Su} (13.6 nM) (FIG. 4d); but they had 1.9-8.2 times higher chemiluminescence QYs (BOPD_{Su}: 0.189; BTPD_{Su}: 0.137; and MBPD_{Su}: 0.045) than that of MPD_{Su} (0.023) (Table 1). Such O₂^{•-}-mediated activation was further verified by HPLC, showing the presence of new peaks at 8.6, 8.4 and 12.1 min which are assigned to the corresponding products from BOPD_{Su}, BTPD_{Su} and MBPD_{Su}, respectively (FIG. 5).

TABLE 1
Photophysical properties of probes
(BOPD_{Su}, BTPD_{Su}, and MBPD_{Su}).

| Sample | Φ_{CI} (einsteins/mol) | Activated probe | Φ_{FI} (%) |
|---|---|---|---|
| BOPD_{Su} | 0.189 | ABOPD | 33.0 |
| BTPD_{Su} | 0.137 | ABTPD | 24.1 |
| MBPD_{Su} | 0.045 | AMBPD | 7.5 |
| MPD_{Su} | 0.023 | AMPD | 3.1 |

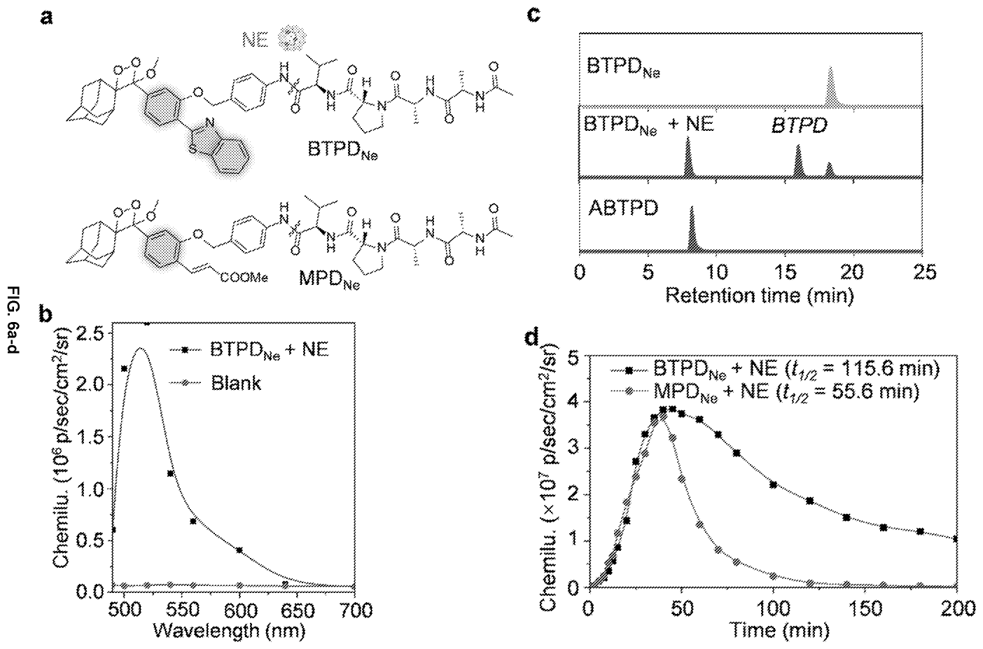

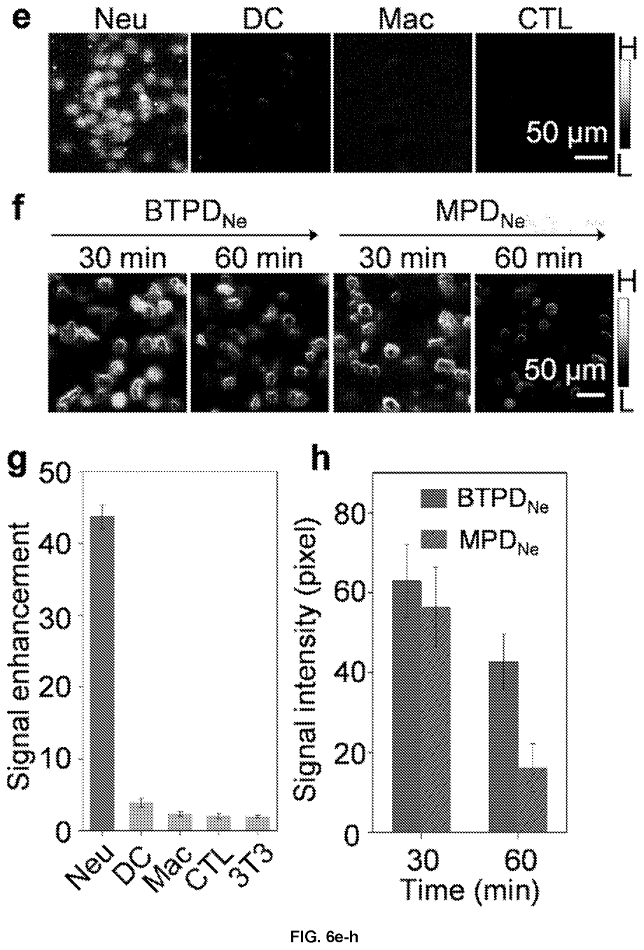

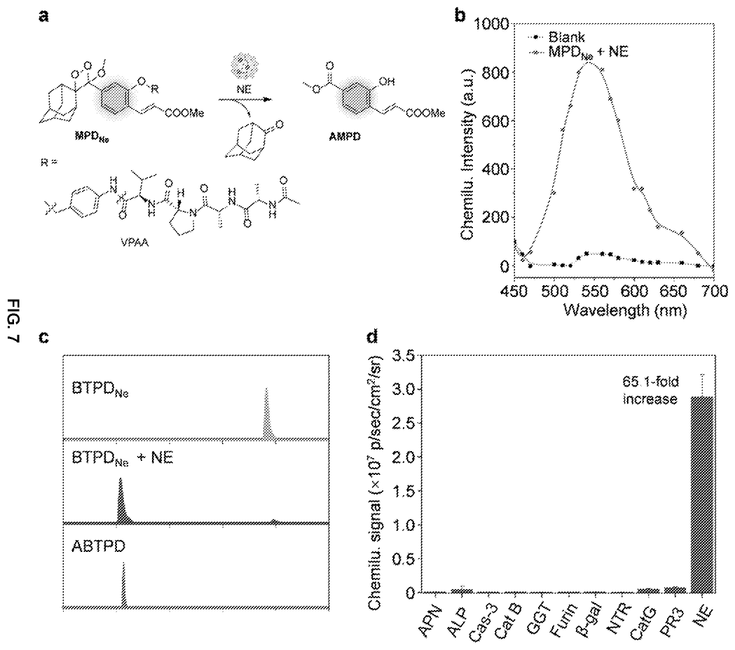

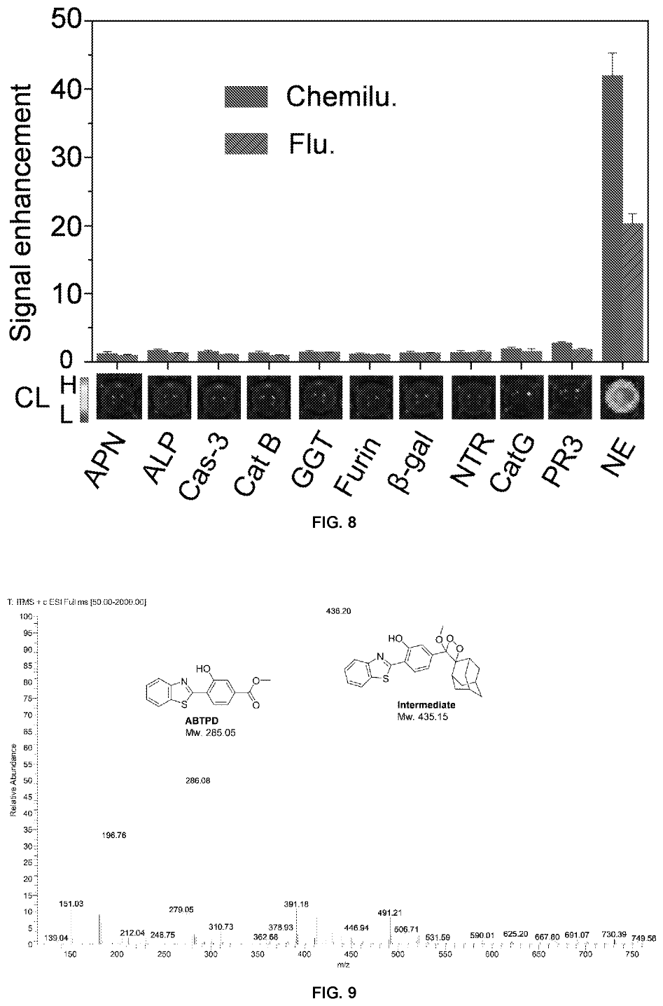

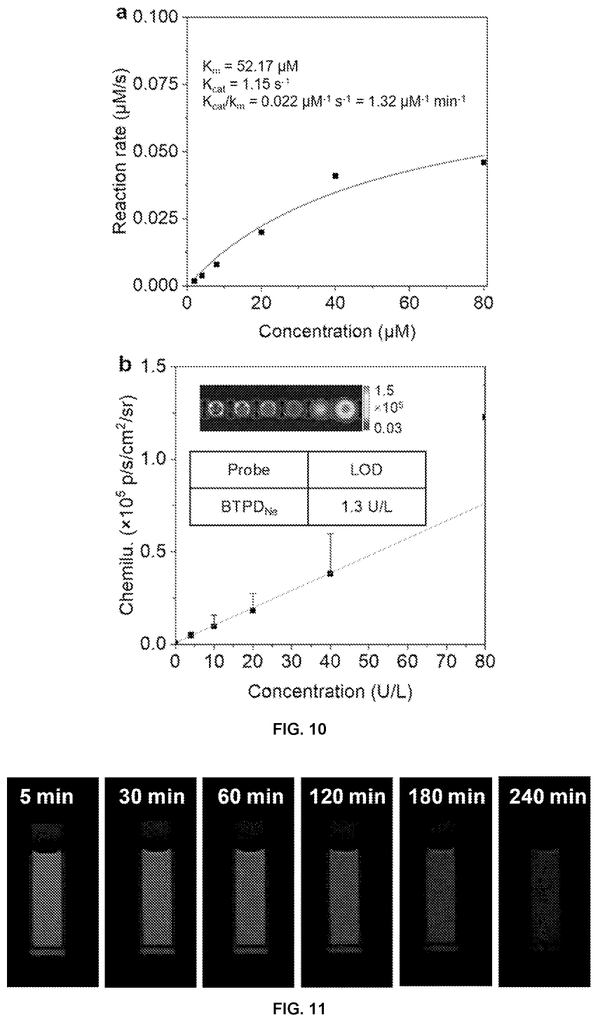

After incubation with NE, BTPD_{Ne} showed ˜42.3-fold chemiluminescence enhancement and 20.1-fold fluorescence increase at 515 nm (FIGS. 6b and 8), while MPD_{Ne} showed 65.1-fold increase chemiluminescence at 550 nm (FIG. 7). The mechanism of NE-triggered chemiluminescence was verified by HPLC (FIG. 6c). After incubating BTPD_{Ne} with NE, HPLC analysis showed an additional peak at 15.9 min. LC-MS analysis confirmed its identity as the uncaged intermediate, adamantylidene-1,2-dioxetane (FIG. 9). As for MPD_{Ne}, the methyl acrylate-phenoxyl-dioxetane intermediate was not observed after NE uncaging due to its instability (FIG. 7c). Subsequent time-dependent chemiluminescence intensity studies (FIG. 6d) further revealed that the half-life of BTPD_{Ne} (˜115.6 min) outperformed MPD_{Ne} (˜ 55.6 min) after NE activation. Both BTPD_{Ne} and MPD_{Ne} showed high selectivity towards NE over other enzymes such as APN, ALP, Cas-3, cat B, GGT, furin, β-gal, NTR, PR3 and CatG (FIGS. 8 and 7d). Furthermore, BTPD_{Ne} exhibited low LOD at ˜1.3 U/L (˜62 ng/mL) and a high enzyme kinetic coefficient of 1.32 μM⁻¹ min⁻¹ (FIG. 10, Cao, T. et al., Anal. Chim. Acta 2020, 127, 295-302; Liu, S. et al., Anal. Chem. 2019, 91, 3877-3884; Hsu, C. et al., Front. Immunol. 2020, 11, 574839; and Zhao, H. et al., Mol. Cancer Ther. 2017, 16, 1866-1876).

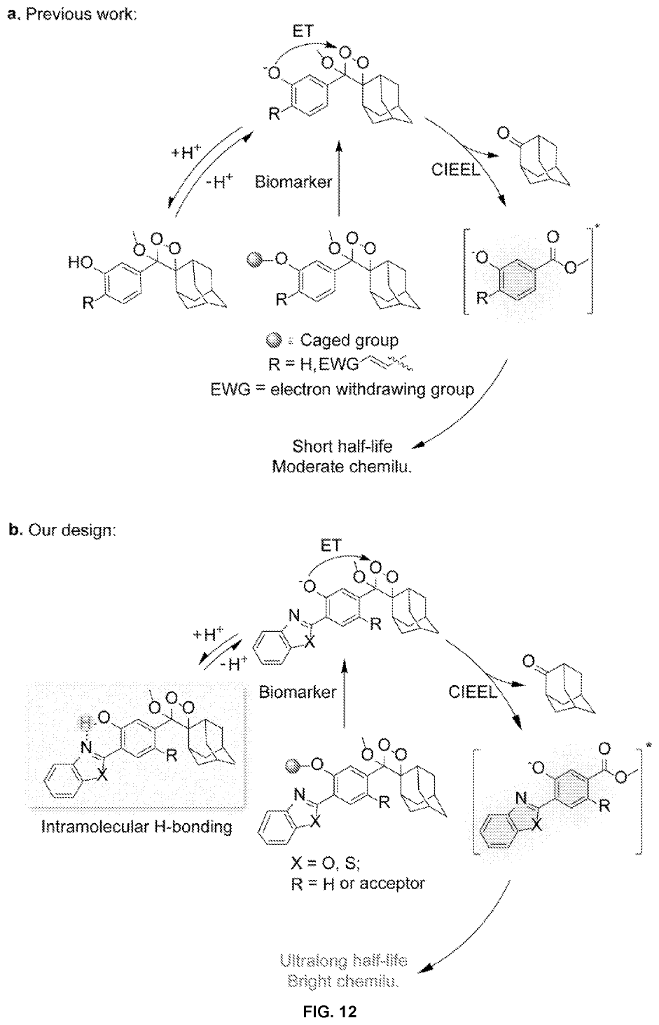

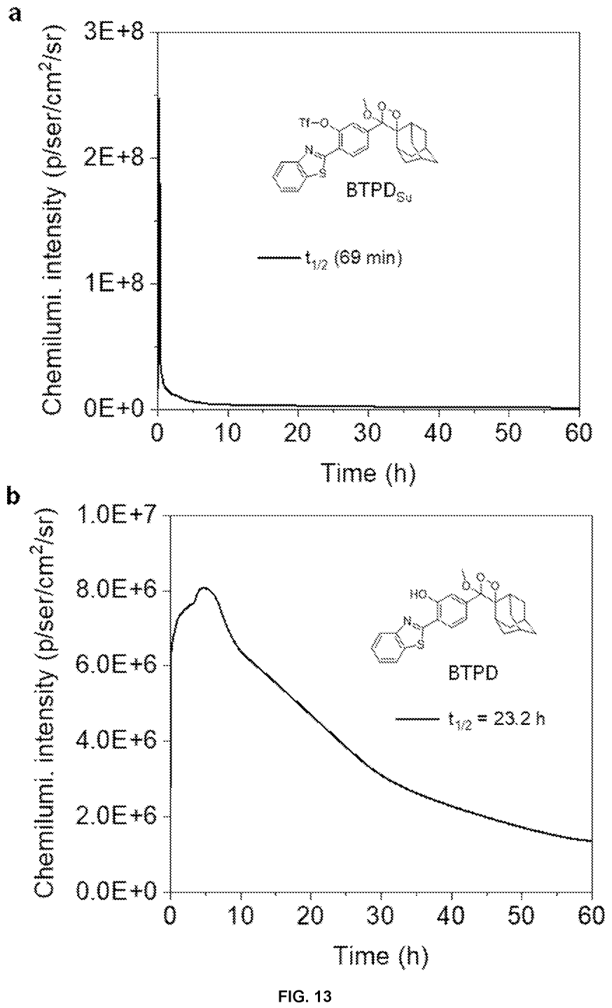

Comparison with the reported methyl acrylate-phenoxyl-dioxetane (MPD_{Su}) revealed that the chemiluminescence half-lives of BOPD_{Su}, BTPD_{Su} and MBPD_{Su} were prolonged to 120 min (33-fold longer than that of the classical counterpart MPD_{Su}, 5.7 min) and the aqueous chemiluminescence QYs were increased to 0.189 einsteins/mol (8.2-fold higher that of MPD_{Su}, 0.023). Chemiluminescent half-lives of BOPD_{Su} (129 min), BTPD_{Su} (121 min) and MBPD_{Su} (132 min) were more than 33 folds longer than that of MPD_{Su} (3.6 min). Even after 4 h incubation with O₂^{•-}, the signal of BTPD_{Su} was still observable by naked eye (FIG. 11). It was hypothesized that the intramolecular hydrogen bonding in BAPDs resulted in the prolonged chemiluminescence (FIG. 12). ¹H NMR was thus used to analyze intramolecular H-bonding for the model molecules: ABTPD and AMPD (FIG. 4e). Titration experiments were conducted in CD₃CN via adding DMSO-de, which is a good hydrogen bond acceptor for disrupting intramolecular interactions (Dhanishta, P. et al., RSC Adv. 2018, 8, 11230-11240). In pure CD₃CN, the proton peak of phenolic hydroxyl in ABTPD was at 12.56 ppm, obviously higher than that for AMPD at 9.37 ppm (FIG. 4e) because H-bonding had a good shielding effect. Upon addition of DMSO-de, the proton peak of phenolic hydroxyl in ABTPD was only shifted by ˜0.17 ppm; for AMPD, the shift was much more significant (˜0.91 ppm) (FIG. 4f). This proved that ABTPD had strong intramolecular H-bonding that was not disrupted in 10% DMSO-d₆/90% CD₃CN. Therefore, NMR analysis and in vitro time-course results confirmed ABTPD set a new record for longest half-life (˜23.2 h, FIG. 13 and Table 2) compared to previously reported phenoxyl-dioxetane luminophores (Table 2), attributed to intramolecular H-bonding of the phenolic hydroxyl group. Similarly, the half-lives of caged BTPD_{Su} also outperformed other activatable chemiluminophores when measured under similar test conditions (Table 3, Cao, J. et al., Chem. Sci. 2018, 9, 2552-2558; Hananya, N. et al., Chem. Eur. J. 2019, 25, 14679-14687; Green, O. et al., ACS Cent. Sci. 2017, 3, 349-358; and An, W. et al., Angew. Chem. 2019, 131, 1375-1379).

TABLE 2
Summary of uncaged dioxetane luminophores with different half-lives (1-14: uncaged probes; and 15-20 caged probes after activation).

| | | | | CL | | |
|---|---|---|---|---|---|---|
| | | | Caged | Emission | | Test |
| Entry | Reference | Chemical Structure | Group | (nm) | t_{1/2} | Conditions |
| 1 | Chem. Eur. J. 2019, 25, 14679-14687 | 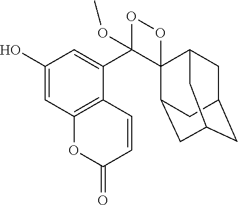 [SMILES: COC1(c2cc(O)cc3oc(=O)ccc23)OOC12C1CC3CC(C1)CC2C3] | No | 465 | ~16 h | in PBS (10% DMSO), pH 7.4, at 37° C. |
| 2 | Tetrahedron. Lett. 1987, 28, 935-938 | 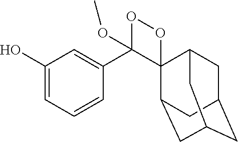 [SMILES: COC1(c2cccc(O)c2)OOC12C1CC3CC(C1)CC2C3] | No | ~460 | ~5.1 years | in o-Xylene at 25° C.^{a} |
| 3 | ACS Cent. Sci. 2017, 3, 349-358; and Chem. Sci. 2019, 10, 1380-1385 | 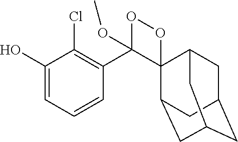 [SMILES: COC1(c2cccc(O)c2Cl)OOC12C1CC3CC(C1)CC2C3] | No No | 470 470 | 17 min 200 s | in PBS (5% DMSO), pH 7.4, 37° C. in PBS (5% DMSO), pH 10, 37° C. |
| 4 | ACS Cent. Sci. 2017, 3, 349-358; and Chem. Sci. 2019, 10, 1380-1385 | 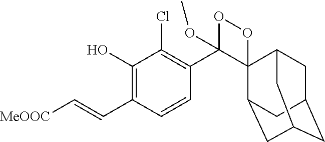 [SMILES: COC(=O)/C=C/c1ccc(C2(OC)OOC23C2CC4CC(C2)CC3C4)c(Cl)c1O] | No No | 540 550 | ~460 s 420 s | in PBS (10% DMSO), pH 7.4, 37° C. in PBS (10% DMSO), pH 10, 37° C. |
| 5 | ACS Cent. Sci. 2017, 3, 349-358 | 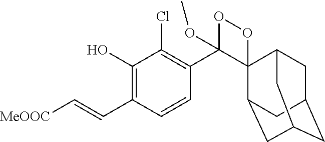 [SMILES: COC(=O)/C=C/c1ccc(C2(OC)OOC23C2CC4CC(C2)CC3C4)c(Cl)c1O] | No | 540 | 23 min | in PBS (5% DMSO), pH 7.4, 37° C. |
| 6 | ACS Cent. Sci. 2107, 3, 349-358 | 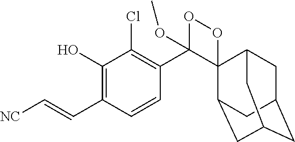 [SMILES: COC1(c2ccc(/C=C/C#N)c(O)c2Cl)OOC12C1CC3CC(C1)CC2C3] | No | 525 | 22 min | in PBS (5% DMSO), pH 7.4, 3.7 ° C. |
| 7 | ACS Cent. Sci. 2017, 3, 349-358; and Chem. Sci. 2019, 10, 1380-1385 | 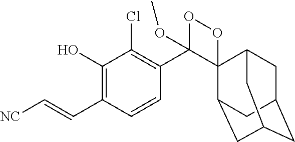 [SMILES: COC1(c2ccc(/C=C/C#N)c(O)c2Cl)OOC12C1CC3CC(C1)CC2C3] | No No | 525 525 | 1100 s 960 s | in PBS (10% DMSO), pH 7.4, 37° C. in PBS (10% DMSO), pH 10, 37° C. |
| 8 | ACS Cent. Sci. 2017, 3, 349-358; and Chem. Sci. 2019, 10-1380-1385 | 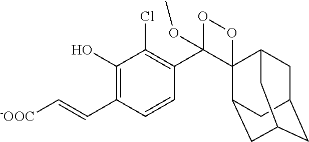 | No No | 510 510 | 85 s 60 s | in PBS (10% DMSO), pH 7.4, 37° C. in PBS (10% DMSO), pH 10, 37° C. |
| 9 | Chem. Sci. 2019, 10, 1380- 1385 | 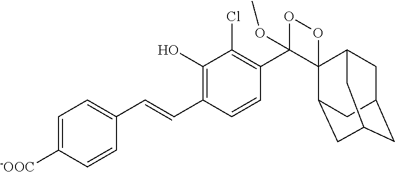 | No No | 535 535 | 8.4 s 7.0 s | in PBS (10% DMSO), pH 7.4, 37° C. in PBS (10% DMSO), pH 10, 37° C. |
| 10 | Chem. Sci, 2019, 10, 1380- 1385 | 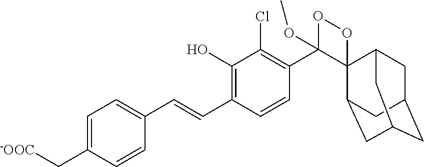 | No No | 500 500 | 5.1 s 4.2 s | in PBS (10% DMSO), pH 7.4, 3.7° C. in PBS (10% DMSO), pH 10, 37° C. |
| 11 | Chem. Sci. 2019, 10, 1380- 1385 | 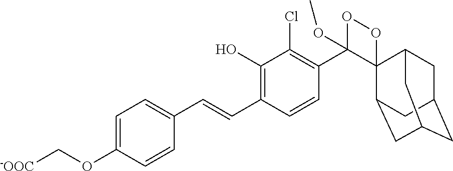 | No No | 490 490 | 3.2 s 2.7 s | in PBS (10% DMSO), pH 7.4, 37° C. in PBS (10% DMSO), pH 10, 37° C. |
| 12 | J. Am. Chem. Soc. 2017, 139, 37, 13243-13248 | 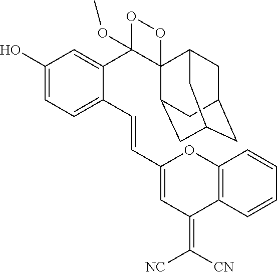 [SMILES: COC1(c2cc(O)ccc2/C=C/C2=CC(=C(C#N)C#N)c3ccccc3O2)OOC12C1CC3CC(C1)CC2C3] | No | 660 | 178 min | in PBS, pH 7.4, 10% FBS |
| 13 | J. Am. Chem. Soc. 2017, 139, 37, 13243-13248 | 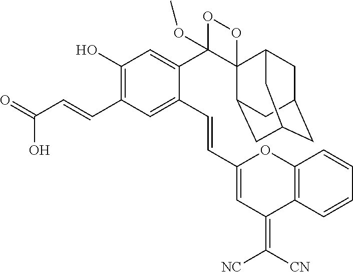 [SMILES: COC1(c2cc(O)c(/C=C/C(=O)O)cc2/C=C/C2=CC(=C(C#N)C#N)c3ccccc3O2)OOC12C1CC3CC(C1)CC2C3] | No | 690 | 53 min | in PBS, pH 7.4, 10% FBS |
| 14 | Our Work | 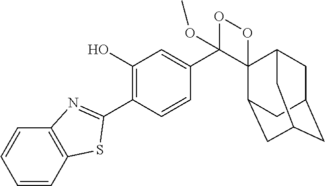 [SMILES: COC1(c2ccc(-c3nc4ccccc4s3)c(O)c2)OOC12C1CC3CC(C1)CC2C3] | No | ~515 | 23.2 h | in PBS (10% DMSO) at 37° C. |

^{a}Calculated from the Arrhenius plots.

TABLE 3
Summary of caged dioxetane luminophores with different half-lives after activation.

| | | | | CL | | |
|---|---|---|---|---|---|---|
| | | | Caged | Emission | | Test |
| Entry | Reference | Chemical Structure | Group | (nm) | t_{1/2} | Conditions |
| 1 | Chem. Commun, 2011, 47, 6713-6715 | 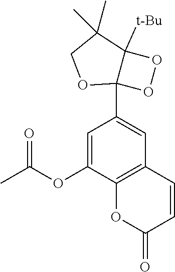 [SMILES: CC(=O)Oc1cc(C23OCC(C)(C)C2(C(C)(C)C)OO3)cc2ccc(=O)oc12] | 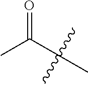 [SMILES: *C(C)=O] | 470 | ~230 min | in DMSO with 170 μM TBAF at 25° C. |
| 2 | Angew. Chem. Int. Edit. 2019, 58, 1361-1365 | 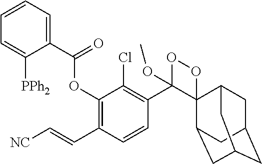 | 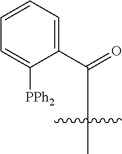 | 525 | ~60 min | in 20 mM HEPES or PBS (pH 7.4), 1% DMSO |
| 3 | Chem. Sci. 2018, 9, 2552-2558 | 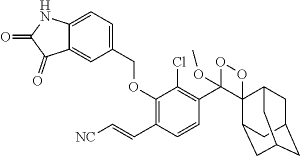 [SMILES: COC1(c2ccc(/C=C/C#N)c(OCc3ccc4c(c3)C(=O)C(=O)N4)c2Cl)OOC12C1CC3CC(C1)CC2C3] | 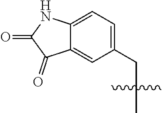 [SMILES: *Cc1ccc2c(c1)C(=O)C(=O)N2] | 525 | ~18 min | in 20 mM HEPES (pH 7.4), 1% DMSO |
| 4 | J. Am. Chem. Soc. 2019, 141, 10581-10584 | 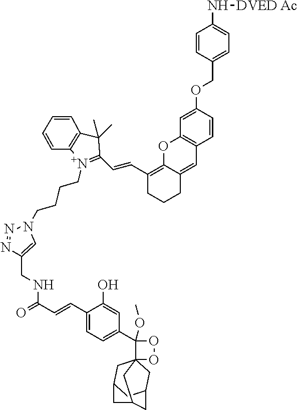 | 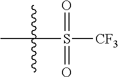 [SMILES: *S(=O)(=O)C(F)(F)F] | 540 | ~3.5 m | in 10 mM PBS buffer (pH = 7.4) |
| 5 | Our Work | 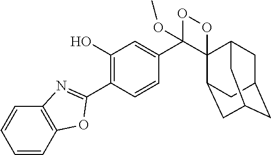 [SMILES: COC1(c2ccc(-c3nc4ccccc4o3)c(O)c2)OOC12C1CC3CC(C1)CC2C3] | 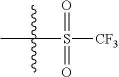 [SMILES: *S(=O)(=O)C(F)(F)F] | 480 | 129 min | in PB (10% DMSO) at 37 |
| 6 | | | 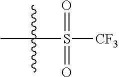 [SMILES: *S(=O)(=O)C(F)(F)F] | 520 | 121 min | in PBS (10% DMSO) at 37° C. |
| | | 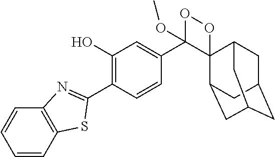 [SMILES: COC1(c2ccc(-c3nc4ccccc4s3)c(O)c2)OOC12C1CC3CC(C1)CC2C3] | 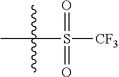 [SMILES: *S(=O)(=O)C(F)(F)F] | 485 | 69 min | in pure DMSO |
| 7 | | 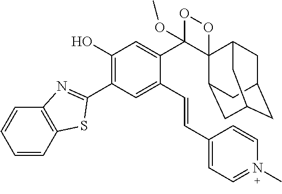 | 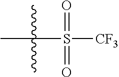 [SMILES: *S(=O)(=O)C(F)(F)F] | 580 | 132 min | in PBS (10% DMSO) at 37° C. |

### Example 5. Effect of pH on Stability

To investigate the effect of pH on stability, half-life and intramolecular hydrogen bonding of BTPD_{Su} and ABTPD, the UV spectra, fluorescence spectra and chemiluminescence signals were measured.

### Stability of BTPD_{Su} and ABTPD at Different pHs

BTPD_{Su} and ABTPD (20 μM) were stored at different pHs (pH 5, 6, 7, 8 and 9) containing 10% DMSO for 2 h. Then, UV spectra of BTPD_{Su} and fluorescence spectra of ABTPD were measured at different pHs.

### Time-Course of BTPD_{Su} at Different pHs

The time-course of BTPD_{Su} (20 μM) was conducted upon the addition of excess O₂^{•-} (40 μM) in different pH buffers with 10% DMSO at 37° C. The chemiluminescence signal was recorded by the microplate reader to calculate half-lives of BTPD_{Su} at different pHs.

### Results and Discussion

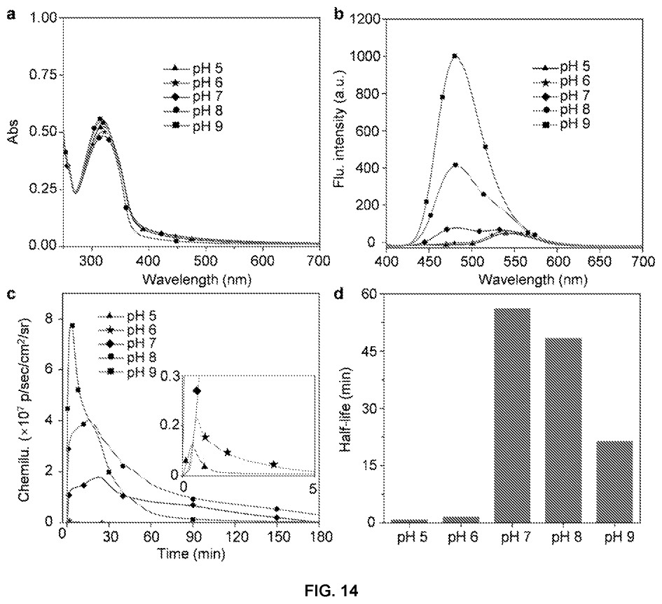

The UV spectra of BTPD_{Su} showed no obvious changes at different pHs, indicating good stability (FIG. 14a). As expected, pH had a slight influence on the intramolecular H-bonding of ABTPD. In alkaline conditions where H-bonding is disrupted, ABTPD showed maximum emission at 481 nm. In acidic conditions where strong H-bonding exists, ABTPD exhibited two emission peaks at 481 and 542 nm based on the excited-state intramolecular proton transfer mechanism (FIG. 14b). The half-life of caged BTPD_{Su} still reached up to ˜56.1 and 48.5 min at pH 7 and 8, respectively, near physiological pH (FIG. 14c-d). These half-lives are comparable to other reported activatable chemiluminophores under physiological conditions. Therefore, BTPD_{Su} still can perform well as a NE-activatable chemiluminescent probe at physiological pH.

### Example 6. Blood Test

### Blood Test

BTPD_{Ne} (20 μM) and MPD_{Ne} (20 μM) were incubated for 30 min in healthy mouse blood (100 μL), respectively. Chemiluminescence intensities were measured by IVIS system bioluminescence with an acquisition time of 60 s.

### Results and Discussion

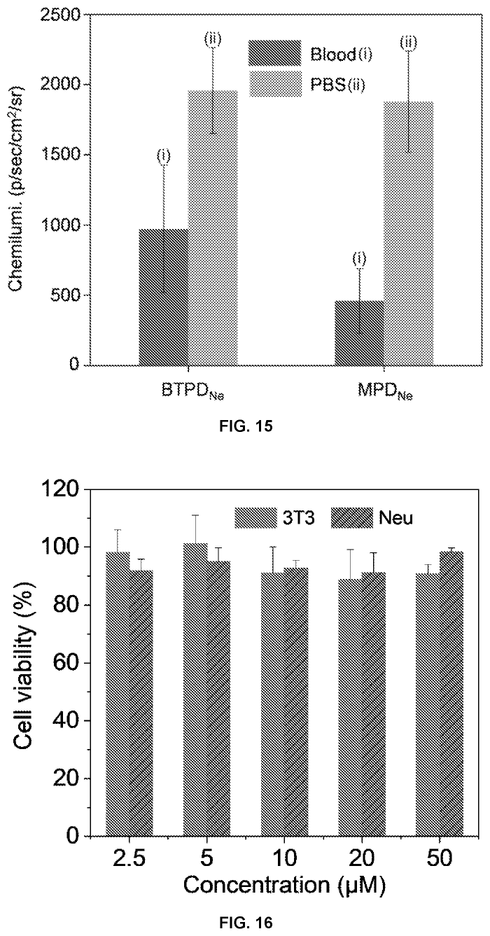

Negligible chemiluminescence intensities were observed for both BTPD_{Ne} and MPD_{Ne} after incubation with healthy mouse blood due to the lower levels of neutrophils (FIG. 15).

### Example 7. Cytotoxicity of BTPD_{Ne}

### Cytotoxicity Assay

Neutrophils and normal mouse embryonic fibroblasts (3T3) cells were seeded in 96-well plates which have 5×10⁴ cells per well and incubated for 24 h. Then, BTPD_{Ne} at different concentrations (2.5, 5, 10, 20 and 50 μM) was added into the neutrophils and 3T3 cells, respectively. After 24 h incubation, MTS assay was added to the cell for 4 h incubation. After incubation, the absorbance of MTS at 490 nm was recorded by using a microplate reader (SpectraMax M, Switzerland). The assays were performed in five sets for each concentration.

### Results and Discussion

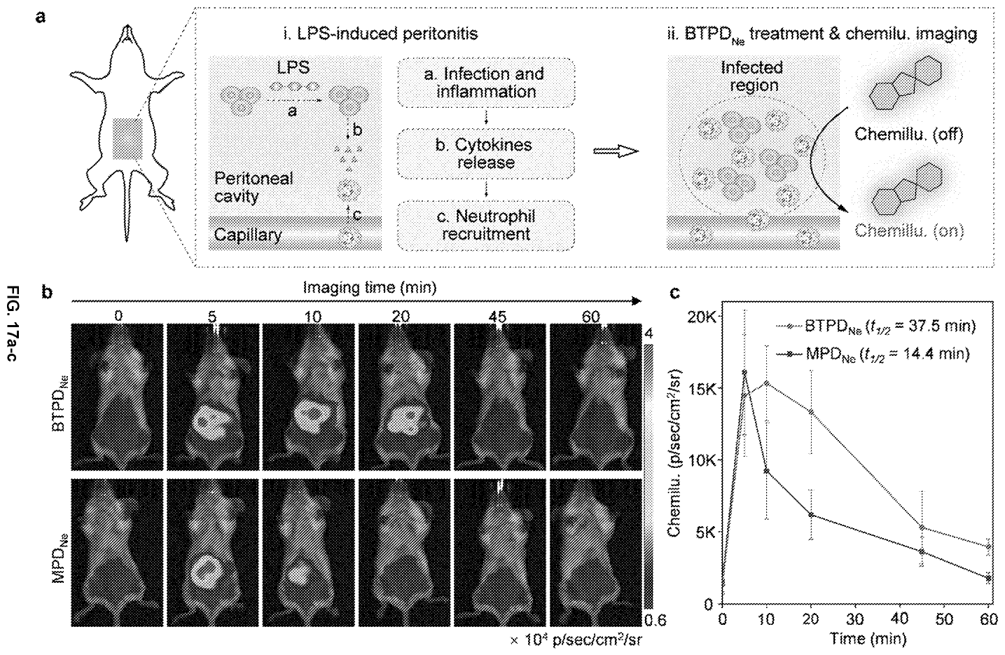

BTPD_{Ne} showed no cytotoxicity against both 3T3 cells and neutrophils at the concentration ranging from 2.5 to 50 μM (FIG. 16). With no cytotoxicity observed in both normal mouse embryonic fibroblasts (3T3) and neutrophils (FIG. 16), BTPD_{Ne} and MPD_{Ne} were next used for cell imaging studies in Example 8.

### Example 8. In Vitro Cell Imaging Studies

### In Vitro Cell Chemiluminescent Imaging Studies

3T3 cells and immune cells (neutrophils, DC cells, T-cells and macrophage) (10⁴ cells) were seeded into confocal cell culture dishes (dia. 15 mm) and incubated for 24 h. Then, the five groups of cells were incubated with BTPD_{Ne} (20 μM in the medium) for 60 min. After incubation, the medium was removed, and the cells were washed thrice. Chemiluminescence imaging of the cells were recorded on LX71 inverted microscope (Olympus), which was equipped with infinity 3-1 (Lumenera) CCD camera. During imaging, the excitation light was blocked and the images were recorded under an open filter, with an acquisition time of 60 s.

### In Vitro Cell Fluorescent Imaging Studies

The preparation process of cell incubation of fluorescence imaging was almost identical to that of in vitro real-time chemiluminescence imaging. Fluorescence imaging of cells was acquired on a Laser Scanning Microscope LSM800 (Zeiss). The excitation and emission wavelengths for cell imaging were 405/480-550 nm for activated ABTPD. Cellular chemiluminescence and fluorescence intensities were quantified by using Image J software.

### Results and Discussion

After incubation with neutrophils for 30 min, the activated chemiluminescence signal of BTPD_{Ne} showed 63.0 pixels (the unit of measurement given by the microscope), which is slightly higher than that of MPD_{Ne} (56.4 pixels) (FIGS. 6f and h). After 1 h of incubation, the signal of MPD_{Ne} quickly decreased to 16.2 pixels which is higher than the background while BTPD_{Ne} still retained 42.7 pixels signal as a result of its long half-life, indicating that the longer half-life of BTPD_{Ne} renders it more suitable for applications in cellular imaging. Therefore, both in vitro enzymatic assay (carried out by following the protocol in Example 4) and cellular imaging results (FIGS. 6d, f and h) confirmed that the half-lives of luminophores can be used as a valid performance indicator of chemiluminescence probes.

### Example 9. LPS-Induced Peritonitis

### LPS-Induced Peritonitis Model

All the animal experiments were conducted and followed according to Care and Use of Laboratory Animals of the Nanyang Technological University-Institutional Animal Care and Use Committee (NTU-IACUC) and the Institutional Animal Care and Use Committee (IACUC) for Animal Experiment, Singapore.

The model was induced by intraperitoneal injection of LPS (15 ng) in 100 μL of PBS or PBS only as a control. Probe BTPD_{Ne} (40 μM·Kg⁻¹) or MPD_{Ne} (40 μM·Kg⁻¹) in PBS (10 mM, pH 7.4) containing 10% DMSO was intraperitoneally injected in mice. Then, chemiluminescent signals at different post-injection timepoints (0, 5, 10, 20, 45 and 60 min) were recorded using IVIS system bioluminescence with acquiring time of 120 s. After 3 h, the peritoneum was flushed with PBS (5 mL)+EDTA (5 mM). After erythrocyte lysis, neutrophils from C57Bl/6 mice were stained with a PE-labeled CD. After treatment with LPS for 3 h, CD11c antibody and an APC-labeled Ly6G antibody were added to the neutrophils, and the neutrophils were analyzed for double-positive events using flow cytometry (BD Biosciences). Data analysis was performed using Flowjo V10. Neutrophils were gated as CD45+CD11b+Ly6G+cells after the exclusion of doublets.

### In Vivo Fluorescence and Chemiluminescence Imaging Studies

Fluorescence and chemiluminescence in vivo imaging of the probes were measured by an IVIS spectrum imaging system. Chemiluminescence imaging of the cells was recorded on LX71 inverted microscope. Tissue slices were cut by Leica, Germany slicer and imaged by a Nikon ECLIPSE 80i microscope. The images of tissue sections and cells were recorded with a LSM800 confocal laser scanning microscope. The white light was provided by 150 W LED High Bay Thermo Light.

### Results and Discussion

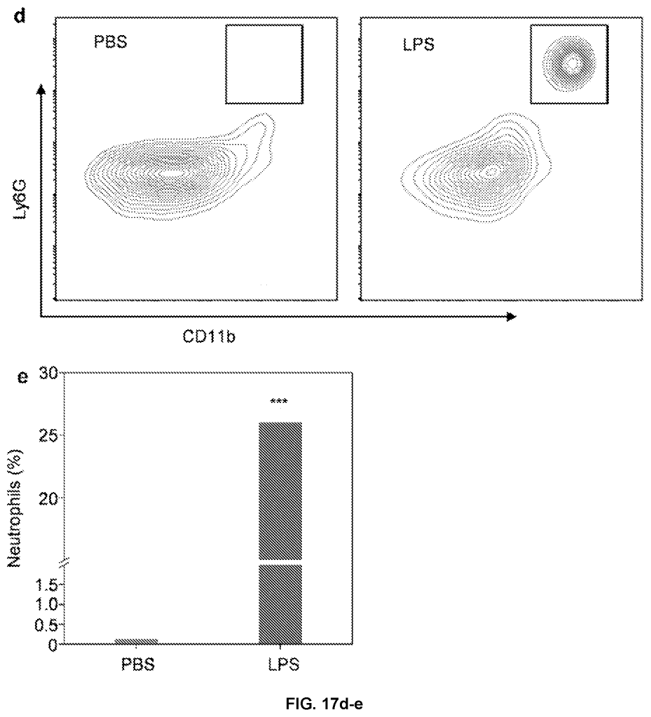

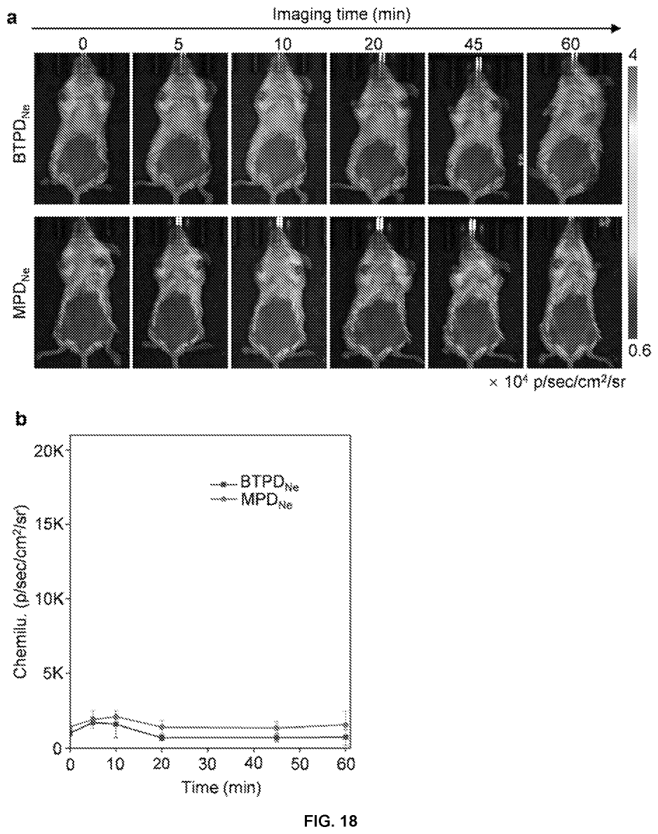

Real-time imaging of neutrophils in LPS-induced peritonitis model was carried out with BTPD_{Ne} and MPD_{Ne}, as a side-by-side comparison. LPS was used to stimulate peritonitis in mice, leading to the activation of CASP4/11 and release of cytokines IL-1p, resulting in neutrophil recruitment (FIG. 17a, B. McDonald et al., Science 2010, 330, 362-366). At 3 h post-treatment of LPS, BTPD_{Ne} or MPD_{Ne} were intraperitoneally injected for in vivo longitudinal tracking of neutrophils. Chemiluminescent signals in the peritoneum of LPS-treated mice intraperitoneally injected with BTPD_{Ne} and MPD_{Ne} increased and reached a maximum at ˜9.2 and 8.5-fold enhancement, respectively, relative to the negative control group (FIG. 18). For BTPD_{Ne} injected mice, chemiluminescent signals showed ˜37.5 min half-life longer than that of MPD_{Ne}-injected mice (˜14.4 min) (FIG. 17). At 20 min post-treatment of probes, a strong chemiluminescent signal from BTPD_{Ne}-injected mice was observed, which is ˜2.15-fold stronger than that of MPD_{Ne}-injected mice (FIG. 17b). Even after 60 min, chemiluminescent signals could still be observed for BTPD_{Ne}-injected mice but not for MPD_{Ne}-treated mice. Therefore, due to the higher brightness and longer half-life, BTPD_{Ne} is more suitable than the classical chemiluminescent probe (MPD_{Ne}) for real-time longitudinal imaging of neutrophils.

### Example 10. Real-Time Imaging for Imiquimod (IMQ)-Induced Psoriasis Models

BTPD_{Ne} was further used for in vivo longitudinal tracking of neutrophils in a murine model of IMQ-induced psoriasis. Fluorescence and chemiluminescence in vivo imaging of the probes were performed by following the protocol in Example 9.

### Real-Time Imaging for IMQ-Induced Psoriasis Models

All the animal experiments were conducted and followed according to Care and Use of Laboratory Animals of the NTU-IACUC and the IACUC for Animal Experiment, Singapore.

BALB/c mice (5 weeks old, female) were divided into three groups including control group, IMQ-treatment group and inhibitor CsA group, and then shaved for an area of 4 cm×3 cm from the backs of mice. The control group was treated with Vaseline (50 mg/d) and the IMQ-treatment group was applied with IMQ cream (60 mg/d, 5%) once a day. The inhibitor group was intraperitoneally injected with CsA (20 mg kg¹) once a day after IMQ cream was applied on the skin of mice for 30 min. Probe BTPD_{Ne} (10 uM, 1 mM in DMSO) was thoroughly mixed with Aquaphor (˜10 mg) and applied for psoriasis imaging, followed by poly(methyl methacrylate) microneedle treatment for 1 min. Then, chemiluminescent signals at different time-points (0, 3, 5, 10, 15, 30, 45 and 60 min) after probe-treatment were recorded using IVIS system bioluminescence with acquiring time of 180 s. Until the third day, all the mice were sacrificed to collect tissue and blood for further experiments (hematoxylin and eosin (H&E) staining and flow cytometry analysis) as described in Example 11.

### Results and Discussion

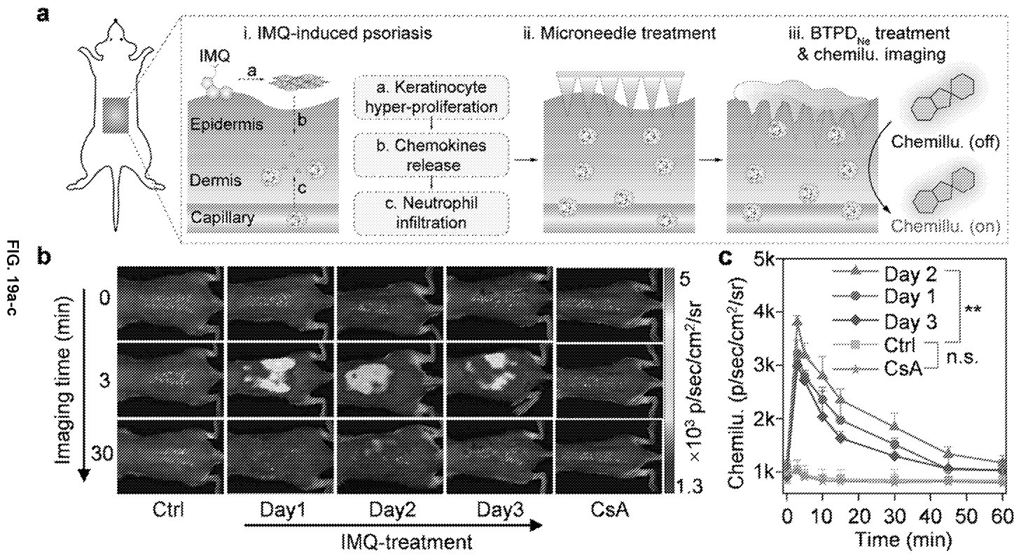

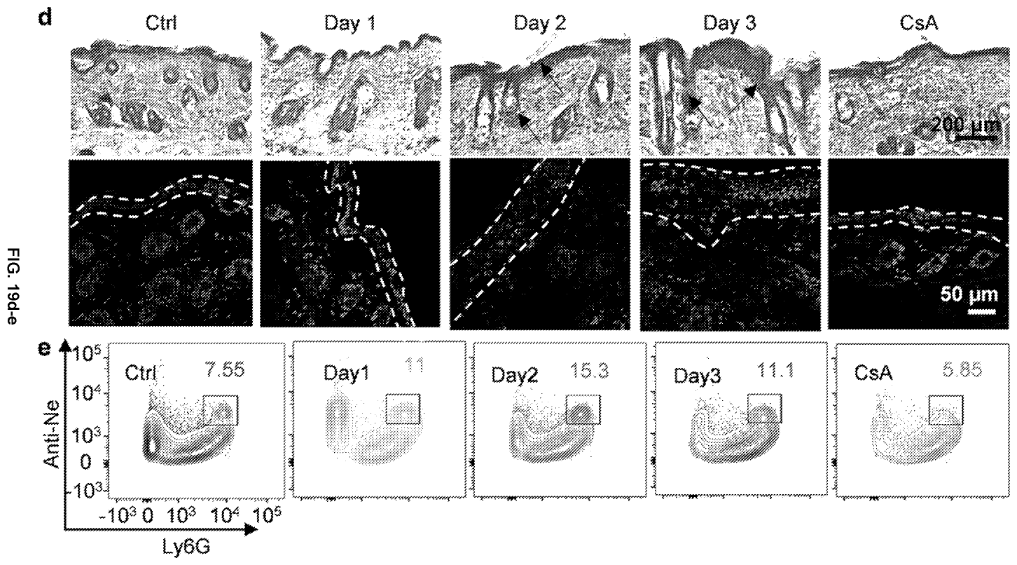

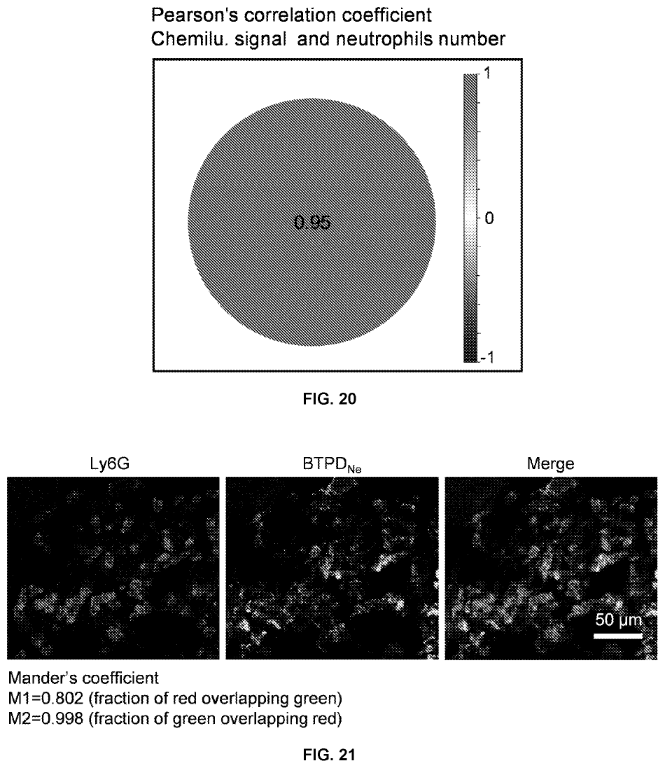

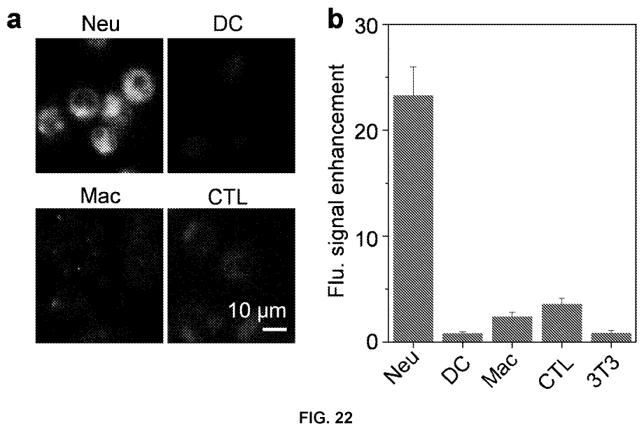

As an immune activator, IMQ stimulates the skin of mice to induce pyroptosis of keratinocytes, releasing cytokines such as pro-1L-1α, CXCL1, and S100A8/A9 (Walter, A. et al., Nat. Commun. 2013, 4, 1560; and Flutter, B. & Nestle, F. O., Eur. J. Immunol. 2013, 43, 3138-3146), leading to neutrophils migration and infiltration (FIG. 19a). After dermal topical treatment with IMQ, BTPD_{Ne} was applied for psoriasis imaging with the assistance of poly(methyl methacrylate) microneedle treatment. At day 1, 2 and 3 post-treatment of IMQ, chemiluminescent signals from dorsal skins gradually increased and reached a maximum at 3 min post-topical administration of BTPD_{Ne}, which are 3.1-, 3.7-, and 2.9-fold higher than the control group, respectively. However, the chemiluminescence signal decreased to background level when the mice were treated with CsA, an immunosuppressant drug (FIG. 19b-c), after IMQ-treatment (Wong, R. L., Winslow, C. M. & Cooper, K. D., Immunol. Today 1993, 14, 69-74), suggesting IMQ-mediated psoriasis could be remarkably inhibited by immunosuppressant. Dorsal skins from mice after different treatments were collected for histological and cellular analysis. IMQ-treated skins displayed increased thickness and neutrophil (CD11c+, Ly6G+) infiltration (FIG. 19d), indicating that the formation of psoriasis was associated with neutrophils after IMQ stimulation (Walter, A. et al., Nat. Commun. 2013, 4, 1560). However, CsA-treated skins showed similar thickness and neutrophil population compared to the skins of the control group. Moreover, flow cytometry analysis in FIG. 19e revealed that IMQ elicited the highest population of NE-expressing neutrophils in skins at day 2, which is 2.03 and 2.62 times higher than that of the control and the CsA-treated group, respectively. This is consistent with the in vivo chemiluminescence signals of BTPD_{Ne} (FIG. 19b), showing a good Pearson's correlation coefficient (0.95) in FIG. 20.

### Example 11. Histopathologic and Immunohistochemical Studies

### Histopathologic and Immunohistochemical Experiments

The dorsal skin samples were separated and soaked in 4% paraformaldehyde (4% PFA) for 12 h and immersed in 30% sucrose solution. The skin samples were stained by hematoxylin and eosin (H&E). Then, the samples were flash-frozen by liquid nitrogen, immobilized with optimal cutting temperature compound, and section slices (5-15 μM). The thickness of epidermis was observed under a microscope (Nikon ECLIPSE 80i microscope). APC-Ly6G, as a neutrophil marker, was used to stain neutrophil cells and DAPI was applied for cell nucleus staining. The immunofluorescence was observed by Nikon ECLIPSE 80i microscope.

After performing in vivo chemiluminescence imaging of neutrophils using BTPD_{Ne} in psoriasis-bearing mice with 2 day IMQ treatment, fresh section slices from dorsal skins were stained with Ly6G. Immunofluorescence imaging of slices with activated BTPD_{Ne} in green color was observed upon excitation at 405 nm and emission at 500-550 nm, while red color from Ly6G was observed upon excitation from 633-647 nm and emission at 660 nm. The immunofluorescence imaging was acquired by Nikon ECLIPSE 80i microscope.

### Results and Discussion

Histopathological and immunohistochemical results in FIG. 19d showed IMQ-treated skins displayed increased thickness and neutrophil (CD11c+, Ly6G+) infiltration, indicating that the formation of psoriasis was associated with neutrophils after IMQ stimulation. However, CsA-treated skins showed similar thickness and neutrophil population to the skins of control group. Moreover, flow cytometry analysis in FIG. 19e revealed that IMQ elicited the highest population of NE-expressing neutrophils in skins at day 2, which was 2.03 and 2.62 times higher than that of control and CsA-treated group. This was consistent with the chemiluminescence signals from in vivo neutrophil imaging (FIG. 19b). Collectively, these data substantiated BTPD_{Ne} were able to noninvasively monitor neutrophils in living mice. In addition, immunofluorescence staining of fresh slides from dorsal skins of mice bearing psoriasis after 2-day IMQ treatment showed that BTPD_{Ne} activation colocalized well with APC-Ly6G, further proving the detection selectivity of BTPD_{Ne} (FIG. 21).

Taken together, based on benzoazole-phenoxyl-dioxetane substrates, an activatable chemiluminescent probe that specifically turns on its chemiluminescence in the presence of NE was developed and applied for in vitro detection and in vivo tracking of neutrophils in the mouse model of psoriasis, one of prevalent, chronic inflammatory skin diseases. BTPD_{Ne} differentiated neutrophils from other immune cells and its chemiluminescent signal was well correlated with the infiltration of neutrophils in disease sites. Thus, this study not only reveals a general molecular mechanism to improve chemiluminescence performance but also provides a new set of chemiluminescent probes for imaging immune responses.

1. A compound of formula I: 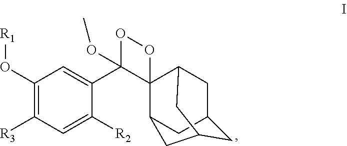
where:
R¹ represents CF₃S(O)₂ or 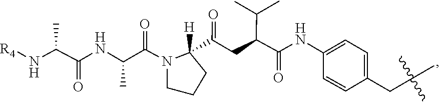
where the wiggly line represents the point of attachment to the rest of the molecule;
R₂ represents H, an acceptor group capable of red-shifting the chemiluminescence emission to the near-infrared region, a polyethylene glycol group, a halogen atom, an electron-withdrawing group or a π* acceptor group capable of accepting electrons;
R₃ represents H, an acceptor group capable of red-shifting the chemiluminescence emission to the near-infrared region, a polyethylene glycol group, a halogen atom, an electron-withdrawing group, a π* acceptor group capable of accepting electrons, or 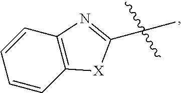
where X represents Se, or more particularly S or O and the wiggly line represents the point of attachment to the rest of the molecule;
R₄ represents 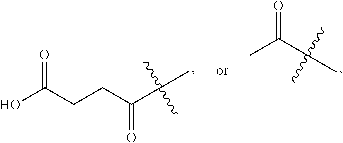
where the wiggly lines represent the point of attachment to the rest of the molecule or
a pharmaceutically acceptable salt or solvate thereof.

2. The compound, or pharmaceutically acceptable salt or solvate thereof, according to claim 1, wherein each acceptor group capable of red-shifting the chemiluminescence emission to the near-infrared region is independently selected from the list of: 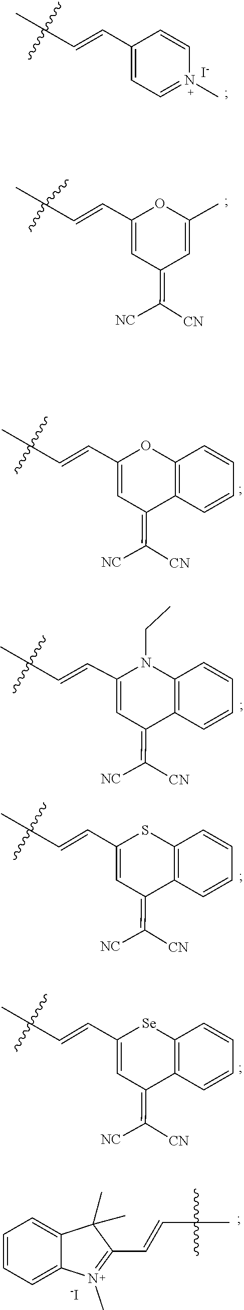 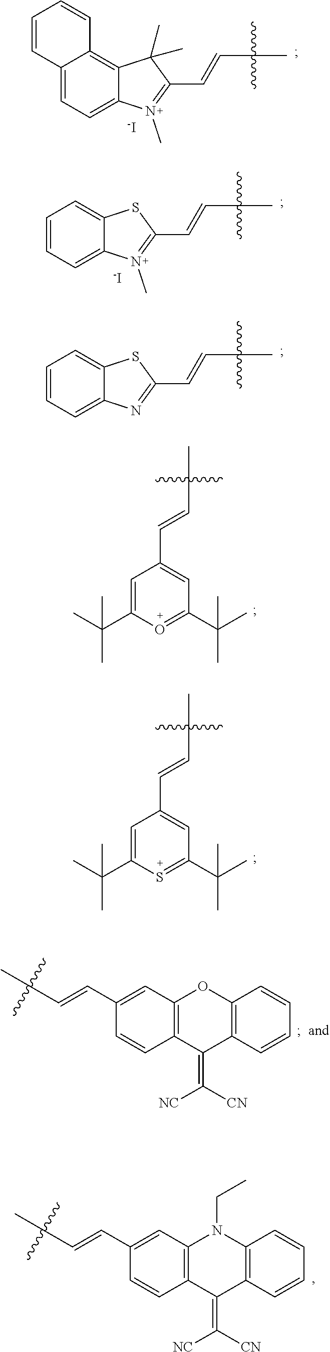
where the wavy line represents the point of attachment to the rest of the molecule.

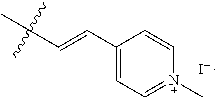

3. The compound, or pharmaceutically acceptable salt or solvate thereof, according to claim 1, wherein each π* acceptor group capable of accepting electrons is selected from the list: 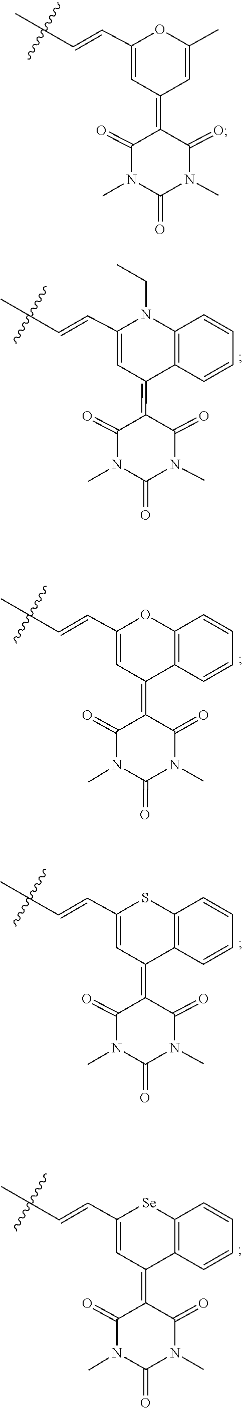 [SMILES: */C=C/C1=CC(=C2C(=O)N(C)C(=O)N(C)C2=O)C=C(C)O1 ; */C=C/C1=CC(=C2C(=O)N(C)C(=O)N(C)C2=O)c2ccccc2N1CC ; */C=C/C1=CC(=C2C(=O)N(C)C(=O)N(C)C2=O)c2ccccc2O1 ; */C=C/C1=CC(=C2C(=O)N(C)C(=O)N(C)C2=O)c2ccccc2S1 ; */C=C/C1=CC(=C2C(=O)N(C)C(=O)N(C)C2=O)c2ccccc2[Se]1] 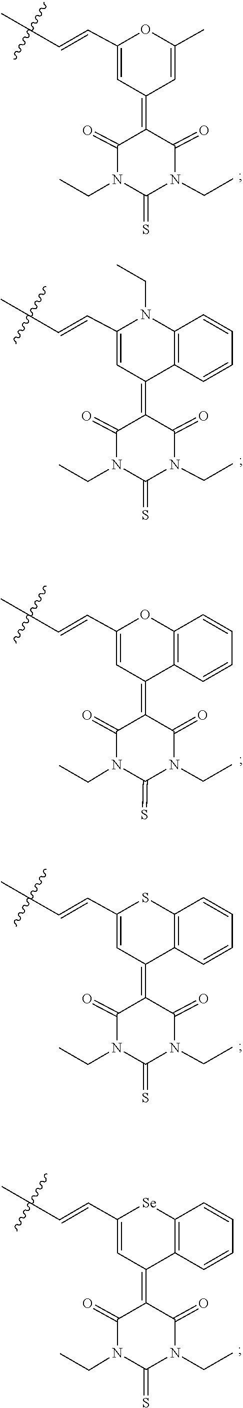 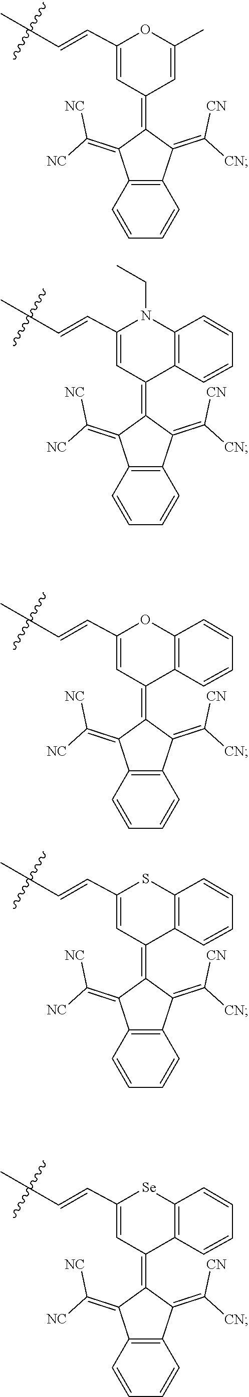 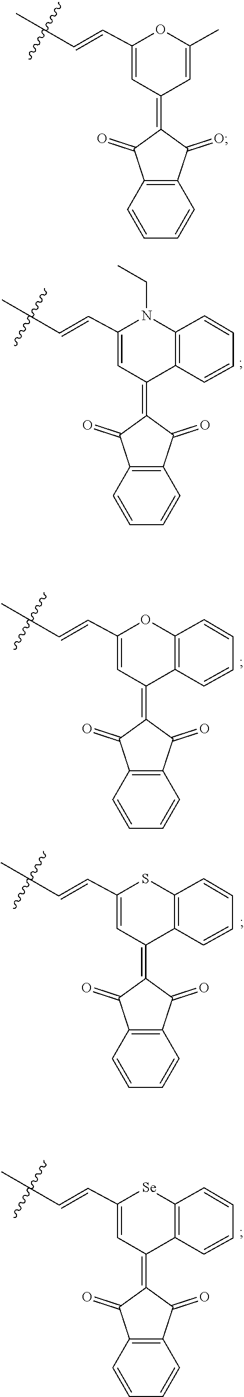 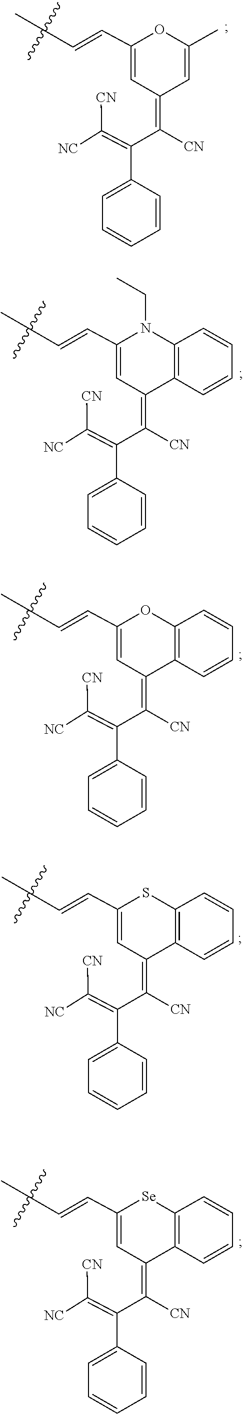 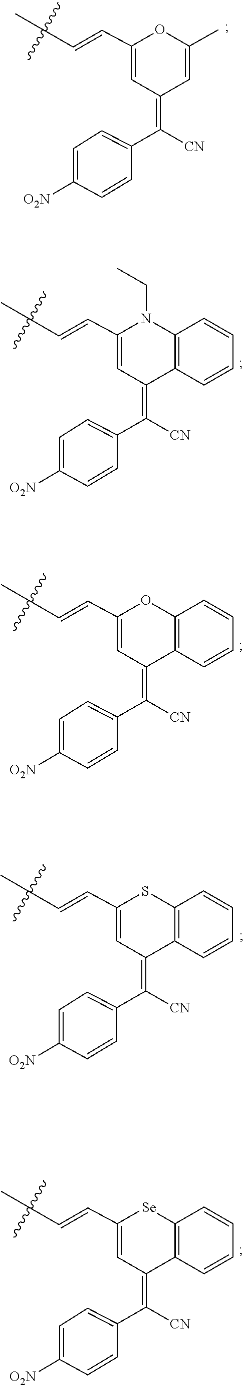 [SMILES: */C=C/C1=CC(=C(/C#N)c2ccc([N+](=O)[O-])cc2)/C=C(C)O1 ; */C=C/C1=CC(=C(/C#N)c2ccc([N+](=O)[O-])cc2)/c2ccccc2N1CC ; */C=C/C1=CC(=C(/C#N)c2ccc([N+](=O)[O-])cc2)/c2ccccc2O1 ; */C=C/C1=CC(=C(/C#N)c2ccc([N+](=O)[O-])cc2)/c2ccccc2S1 ; */C=C/C1=CC(=C(/C#N)c2ccc([N+](=O)[O-])cc2)/c2ccccc2[Se]1] 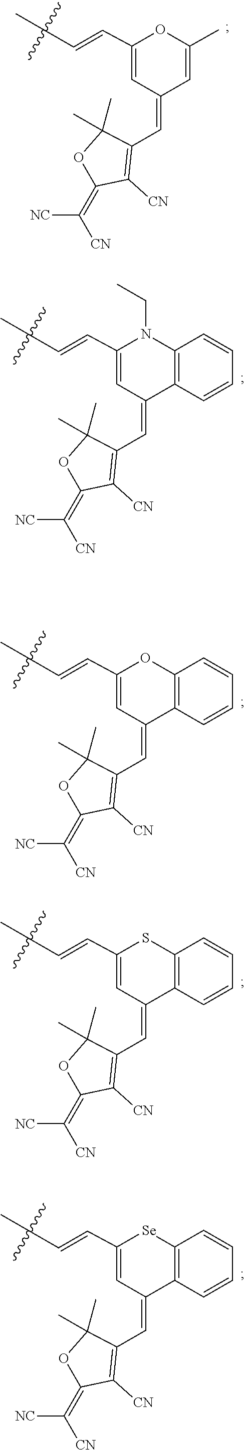 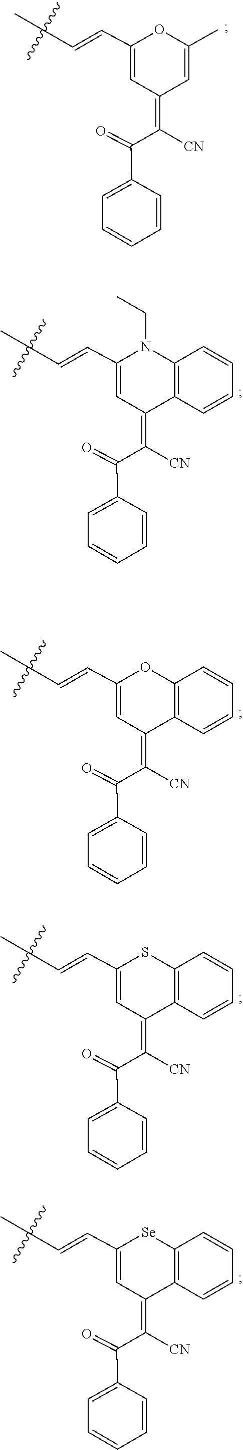 [SMILES: */C=C/C1=CC(=C(/C#N)C(=O)c2ccccc2)/C=C(C)O1 ; */C=C/C1=CC(=C(/C#N)C(=O)c2ccccc2)/c2ccccc2N1CC ; */C=C/C1=CC(=C(/C#N)C(=O)c2ccccc2)/c2ccccc2O1 ; */C=C/C1=CC(=C(/C#N)C(=O)c2ccccc2)/c2ccccc2S1 ; */C=C/C1=CC(=C(/C#N)C(=O)c2ccccc2)/c2ccccc2[Se]1] 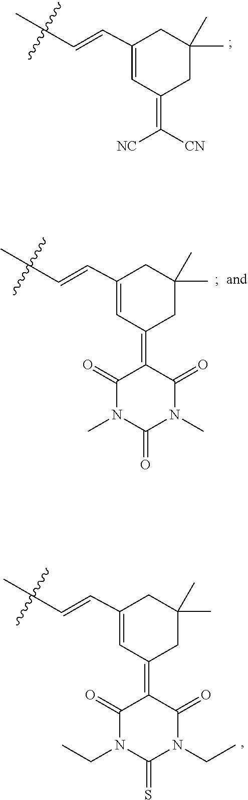 [SMILES: */C=C/C1=CC(=C(C#N)C#N)CC(C)(C)C1 ; */C=C/C1=CC(=C2C(=O)N(C)C(=O)N(C)C2=O)CC(C)(C)C1 ; */C=C/C1=CC(=C2C(=O)N(CC)C(=S)N(CC)C2=O)CC(C)(C)C1]
where the wavy line represents the point of attachment to the rest of the molecule.

4. The compound, or pharmaceutically acceptable salt or solvate thereof, according to claim 1, wherein each electron withdrawing group is selected from the list: 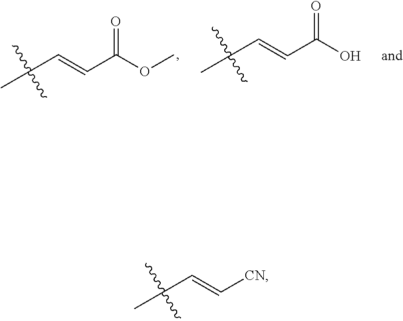
where the wavy line represents the point of attachment to the rest of the molecule.

5. The compound, or pharmaceutically acceptable salt or solvate thereof, according to claim 1, wherein each polyethylene glycol group has the formula: 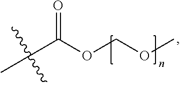
where n is from 1 to 227 and the wiggly line represents the point of attachment to the rest of the molecule.

6. The compound, or pharmaceutically acceptable salt or solvate thereof, according to claim 1, wherein:
R₁ is 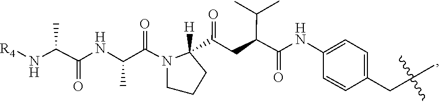
where the wiggly line represents the point o attachment to the rest o the molecule;
R₂ represents H, an acceptor group capable of red-shifting the chemiluminescence emission to the near-infrared region, or a polyethylene glycol group;
R₃ represents 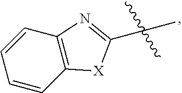
where X represents S or O and the wiggly line represents the point of attachment to the rest of the molecule.

7. The compound according to claim 6, wherein R₁ represents CF₃S(O)₂.

8. The compound according to claim 6, wherein R₂ represents H or

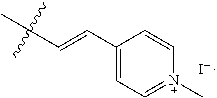

9. The compound, or pharmaceutically acceptable salt or solvate thereof, according to claim 1, wherein:
R¹ represents 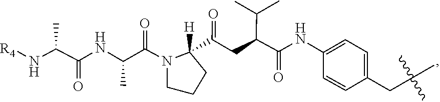
where the wiggly line represents the point of attachment to the rest of the molecule;
R₂ represents H, a halogen atom, an electron-withdrawing group or a π* acceptor group capable of accepting electrons;
R₃ represents H, a halogen atom, an electron-withdrawing group or a π* acceptor group capable of accepting electrons.

10. The compound according to claim 9, wherein R₃ represents H, a halogen atom, or an electron-withdrawing group.

11. The compound, or pharmaceutically acceptable salt or solvate thereof, according to claim 1 wherein the compound is selected from the list:

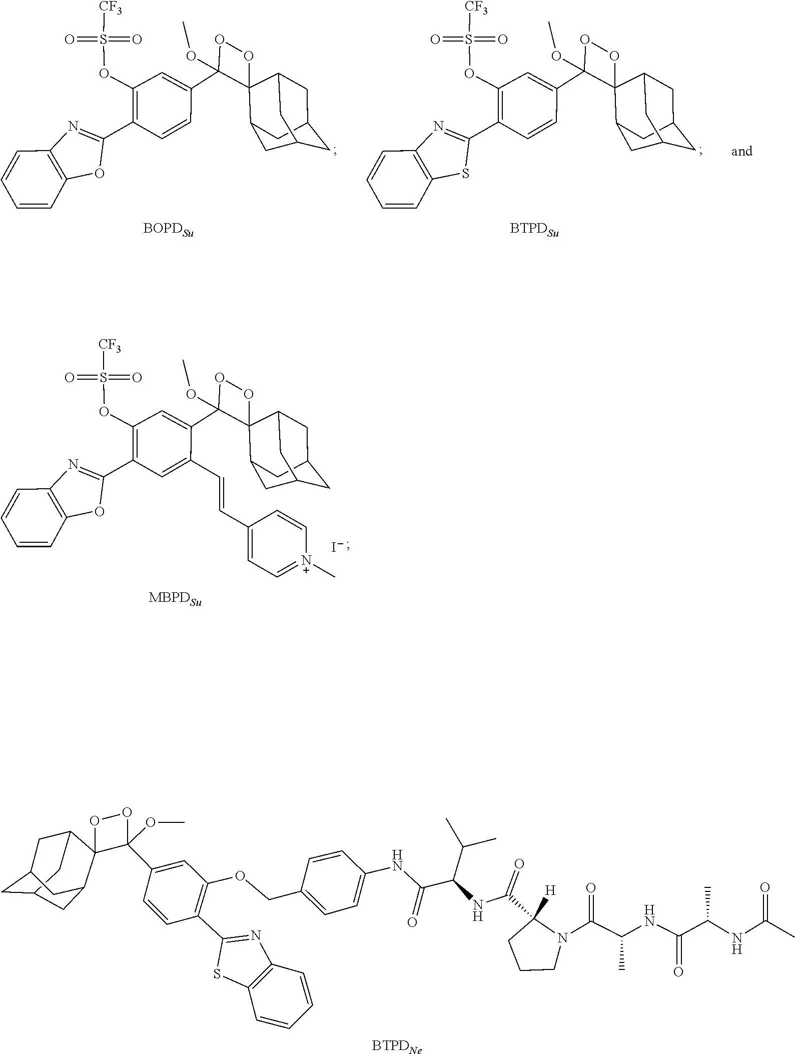

12. A method for detection of neutrophil elastase in an analyte, the method comprising the following steps:
(a) providing an analyte and a fluid comprising a compound of formula I, or pharmaceutically acceptable salt or solvate thereof, according to claim 1;
(b) contacting the analyte with the fluid comprising a compound of formula I, or pharmaceutically acceptable salt or solvate thereof, for a period of time; and
(c) after the period of time detecting any chemiluminescence, wherein the presence of neutrophil elastase in the fluid comprising the analyte and the fluid comprising a compound of formula I, or pharmaceutically acceptable salt or solvate thereof, is indicated by chemiluminescence.

13. A method for detection of neutrophil elastase in vivo, the method comprising the following steps:
(ai) administering a compound of formula I, or pharmaceutically acceptable salt or solvate thereof, according to claim 1 to a subject; and
(aii) detecting any chemiluminescence, wherein the presence of neutrophil elastase in vivo is indicated by chemiluminescence.

14. A method for identifying a compound suitable for the treatment of psoriasis, the method comprising:
(bi) providing a mouse where the skin exhibits neutrophil infiltration;
(bii) contacting the skin with a test material for a first period of time;
(biii) after the first period of time, contacting the drug-treated skin with a compound of formula I, or a pharmaceutically acceptable salt or solvate thereof, as defined in claim 1 for a second period of time; and
(biv) after the second period of time any chemiluminescent signal skin is detected and compared to a blank control, where a reduced quantitative chemiluminescence readout in the presence of the test compound compared to the blank control is indicative of anti-psoriasis activity.

15. A method for identifying a compound suitable for the treatment of peritonitis, the method comprising:
(ci) providing a mouse suffering from peritonitis, where the peritonitis is associated with ascites, where the ascites exhibits neutrophil infiltration (e.g where the mouse's peritonitis was induced by intraperitoneal injection of lipopolysaccharides (LPS));
(cii) contacting an abdomen of the mouse with a test material for a first period of time;
(ciii) after the first period of time, contacting the LPS-treated abdomen with a compound of formula I, or a pharmaceutically acceptable salt or solvate thereof, as defined in claim 1 for a second period of time; and
(civ) after the second period of time any chemiluminescent signal abdomen is detected and compared to a blank control, where a reduced quantitative chemiluminescence readout in the presence of the test compound compared to the blank control is indicative of peritonitis activity, where mice were treated with PBS only.

16. The compound, or pharmaceutically acceptable salt or solvate thereof, according to claim 2, wherein each acceptor group capable of red-shifting the chemiluminescence emission to the near-infrared region is [chemical structure]
